(19) United States
(12) Patent Application Publication
(10) Pub. No.: US 2003/0166176 A1
(43) Pub. Date: Sep. 4, 2003

(54) Taxoid-14-beta-hydroxylases and methods for their use

(75) Inventors: Rodney B. Croteau, Pullman, WA (US); Anne Schoendorf, Collonges Sous Saleve (FR); Stefan Jennewein, Alfdorf (DE)

Correspondence Address: KLARQUIST SPARKMAN, LLP, 121 SW SALMON STREET, SUITE 1600, PORTLAND, OR, 97204, US

(73) Assignee: Washington State University Research Foundation

(21) Appl. No.: 10/356,153

(22) Filed: Jan. 29, 2003

Related U.S. Application Data
(63) Continuation-in-part of application No. 10/142,231, filed on May 8, 2002.
Application No. 10/142,231 is a continuation of application No. PCT/US00/31254, filed on Nov. 13, 2000.
(60) Provisional application No. 60/165,250, filed on Nov. 12, 1999.

(51) Int. Cl. C12P 17/02; C07H 21/04; C12N 9/02; C12P 21/02; C12N 5/04; C 07D 3 5/14

(52) U.S. Cl. 435/123; 435/189; 435/69.1; 435/320.1; 435/419; 536/23.2; 549/510

(57) ABSTRACT

Oxygenase enzymes and the use of such enzymes to produce paclitaxel (Taxol®) related taxoids, as well as intermediates in the Taxol® biosynthetic pathway are disclosed. Also disclosed are nucleic acid sequences encoding the oxygenase enzymes. Particular oxygenase enzymes include the disclosed taxoid-14-beta hydroxylases.

### CROSS REFERENCE TO RELATED APPLICATIONS

This application is a continuation-in-part of, and claims priority to, co-pending U.S. application Ser. No. 10/142,231, filed May 8, 2002, which is a continuation of, and claims priority to, PCT application no. PCT/US00/31254, filed on Nov. 13, 2000, which claims priority to U.S. Provisional Patent Application No. 60/165,250, filed on Nov. 12, 1999. Each of these applications is incorporated by reference herein in its entirety.

### ACKNOWLEDGMENT OF GOVERNMENT SUPPORT

This invention was made with government support under National Cancer Institute Grant No. CA-55254. The government has certain rights in this invention.

### FIELD OF THE INVENTION

The invention relates to enzymes related to Taxol® (paclitaxel), and methods of using such enzymes to produce Taxol® and related taxoids. In particular, this invention relates to oxygenases involved in Taxol® production such as hydroxlases.

### INTRODUCTION

Cytochrome P450

Cytochrome P450 proteins are enzymes that have a unique sulfur atom ligated to the heme iron and that, when reduced, form carbon monoxide complexes. When complexed to carbon monoxide they display a major absorption peak (Soret band) near 450 nm. There are numerous members of the cytochrome P450 group including enzymes from both plants and animals. Members of the cytochrome P450 group can catalyse reactions such as unspecific monooxygenation, camphor 5-monooxygenation, steroid 11β-monooxygenation, and cholesterol monooxygenation (Smith et al. (eds.), Oxford Dictionary of Biochemistry and Molecular Biology, Oxford University Press, New York, 1997).

Paclitaxel

The complex diterpenoid Taxol® (Bristol-Myers Squibb; common name paclitaxel) (Wani et al., J. Am. Chem. Soc. 93:2325-2327, 1971) is a potent antimitotic agent with excellent activity against a wide range of cancers, including ovarian and breast cancer (Arbuck and Blaylock, Taxol: Science and Applications, CRC Press, Boca Raton, 397-415, 1995; and Holmes et al., ACS Symposium Series 583:31-57, 1995). Taxol® was isolated originally from the bark of the Pacific yew (Taxus brevifolia). For a number of years, Taxol® was obtained exclusively from yew bark, but low yields of this compound from the natural source coupled to the destructive nature of the harvest, prompted new methods of Taxol® production to be developed. Taxol® currently is produced primarily by semisynthesis from advanced taxane metabolites (Holton et al., Taxol: Science and Applications, CRC Press, Boca Raton, 97-121, 1995) that are present in the needles (a renewable resource) of various Taxus species. However, because of the increasing demand for this drug both for use earlier in the course of cancer intervention and for new therapeutic applications (Goldspiel, Pharmacotherapy 17:110S-125S, 1997), availability and cost remain important issues. Total chemical synthesis of Taxol® currently is not economically feasible. Hence, biological production of the drug and its immediate precursors will remain the method of choice for the foreseeable future. Such biological production may rely upon either intact Taxus plants, Taxus cell cultures (Ketchum et al., Biotechnol. Bioeng. 62:97-105, 1999), or, potentially, microbial systems (Stierle et al., J. Nat. Prod. 58:1315-1324, 1995). In all cases, improving the biological production yields of Taxol® depends upon a detailed understanding of the biosynthetic pathway, the enzymes catalyzing the sequence of reactions, especially the rate-limiting steps, and the genes encoding these proteins. Isolation of genes encoding enzymes involved in the pathway is a particularly important goal, since overexpression of these genes in a producing organism can be expected to markedly improve yields of the drug.

The Taxol® biosynthetic pathway is considered to involve more than 12 distinct steps (Floss and Mocek, Taxol: Science and Applications, CRC Press, Boca Raton, 191-208, 1995; and Croteau et al., Curr. Top. Plant Physiol. 15:94-104, 1996). However, very few of the enzymatic reactions and intermediates of this complex pathway have been defined. The first committed enzyme of the Taxol® pathway is taxadiene synthase (Koepp et al., J. Biol. Chem. 270:8686-8690, 1995) that cyclizes the common precursor geranylgeranyl diphosphate (Hefner et al., Arch. Biochem. Biophys. 360:62-74, 1998) to taxadiene (FIG. 1). The cyclized intermediate subsequently undergoes modification involving at least eight oxygenation steps, a formal dehydrogenation, an epoxide rearrangement to an oxetane, and several acylations (Floss and Mocek, Taxol: Science and Applications, CRC Press, Boca Raton, 191-208, 1995; and Croteau et al., Curr. Top. Plant Physiol. 15:94-104, 1996). Taxadiene synthase has been isolated from T. brevifolia and characterized (Hezari et al., Arch. Biochem. Biophys. 322:437-444, 1995), the mechanism of action defined (Lin et al., Biochemistry 35:2968-2977, 1996), and the corresponding cDNA clone isolated and expressed (Wildung and Croteau, J. Biol. Chem. 271:9201-9204, 1996).

The second specific step of Taxol® biosynthesis is an oxygenation (hydroxylation) reaction catalyzed by taxadiene-5α-hydroxylase. The enzyme has been demonstrated in Taxus microsome preparations (Hefner et al., Methods Enzymol. 272:243-250, 1996), shown to catalyze the stereospecific hydroxylation of taxa-4(5),11(12)-diene to taxa-4(20),11(12)-dien-5α-ol (i.e., with double-bond rearrangement), and characterized as a cytochrome P450 oxygenase (Hefner et al., Chemistry and Biology 3:479-489, 1996).

Since the first specific oxygenation step of the Taxol® pathway was catalyzed by a cytochrome P450 oxygenase, it was logical to assume that subsequent oxygenation (hydroxylation and epoxidation) reactions of the pathway would be carried out by similar cytochrome P450 enzymes. Microsomal preparations (Hefner et al., Methods Enzymol. 272:243-250, 1996) were optimized for this purpose, and shown to catalyze the hydroxylation of taxadiene or taxadien-5α-ol to the level of a pentaol (see FIG. 2 for tentative biosynthetic sequence and structures based on the evaluation of taxane metabolite abundances (Croteau et al., Curr. Topics Plant Physiol. 15:94-104, 1995), providing evidence for the involvement of at least five distinct cytochrome P450 taxane (taxoid) hydroxylases in this early part of the pathway (Hezari et al., Planta Med. 63:291-295, 1997).

Also, the remaining three oxygenation steps (C1 and C7 hydroxylations and an epoxidation at C4-C20; see FIGS. 1 and 3) likely are catalyzed by cytochrome P450 enzymes, but these reactions reside too far down the pathway to observe in microsomes by current experimental methods (Croteau et al., Curr. Topics Plant Physiol. 15:94-104, 1995; and Hezari et al., Planta Med. 63:291-295, 1997). Since Taxus (yew) plants and cells do not appear to accumulate taxoid metabolites bearing fewer than six oxygen atoms (i.e., hexaol or epoxypentaol) (Kingston et al., Prog. Chem. Org. Nat. Prod. 61:1-206, 1993), such intermediates must be rapidly transformed down the pathway, indicating that the oxygenations (hydroxylations) are relatively slow pathway steps and, thus, important targets for gene cloning.

Isolation of the genes encoding the oxygenases that catalyze the oxygenase steps of Taxol® biosynthesis would represent an important advance in efforts to increase Taxol® and taxoid yields by genetic engineering and in vitro synthesis.

### SUMMARY OF THE INVENTION

This disclosure stems from the discovery of twenty-one amplicons (regions of DNA amplified by a pair of primers using the polymerase chain reaction (PCR)). These amplicons can be used to identify oxygenases, for example, the oxygenases shown in SEQ ID NOS: 56-68 and 87-92, that are encoded by the nucleic acid sequences shown in SEQ ID NOS: 43-55 and 81-86. One particular oxygenase is the taxoid-14β-hydroxylase shown in SEQ ID NOS: 54 and 67.

These sequences were isolated from the Taxus genus, and the respective oxygenases are useful for the synthetic production of Taxol® and related taxoids, as well as intermediates within the Taxol® biosynthetic pathway, and other taxoid derivatives. The sequences also can be used for the creation of transgenic organisms that either produce the oxygenases for subsequent in vitro use, or produce the oxygenases in vivo so as to alter the level of Taxol® and taxoid production within the transgenic organism.

Included are the nucleic acid sequences shown in SEQ ID NOS: 1-21 and the corresponding amino acid sequences shown in SEQ ID NOS: 22-42, respectively, as well as fragments of these nucleic acid sequences and amino acid sequences. These sequences are useful for isolating the nucleic acid and amino acid sequences corresponding to full-length oxygenases. These amino acid sequences and nucleic acid sequences also are useful for creating specific binding agents that recognize the corresponding oxygenases.

Accordingly, oxygenases and fragments of oxygenases that have amino acid and nucleic acid sequences that vary from the disclosed sequences can be identified, such as oxygenase amino acid sequences that vary by one or more conservative amino acid substitutions, or that share at least 50% sequence identity with the amino acid sequences provided, while maintaining oxygenase activity. Nucleic acid sequences encoding the oxygenases and fragments of the oxygenases that maintain taxoid oxygenase and/or CO binding activity can be cloned into vectors using standard molecular biology techniques. These vectors then can be used to transform host cells, including non-human host cells. Thus, a host cell can be modified to express either increased or decreased levels of an oxygenase, such as a taxoid-14β-hydroxylase.

Also included are methods for isolating nucleic acid sequences encoding full-length oxygenases. The methods involve hybridizing at least ten contiguous nucleotides of any of the nucleic acid sequences shown in SEQ ID NOS: 1-21, 43-55, and 81-86 to a second nucleic acid sequence, wherein the second nucleic acid sequence encodes a taxoid oxygenase and/or maintains CO binding activity. In particular, at least ten contiguous nucleotides of SEQ ID NO: 54 can be hybridized to a second nucleic acid sequence that encodes a taxoid oxygenase and/or maintains CO binding activity, such as a second nucleic acid sequence that encodes a taxoid-14β-hydroxylase. This method can be practiced in the context of, for example, Northern blots, Southern blots, and the polymerase chain reaction (PCR).

The disclosed sequences also can be used to add at least one oxygen atom to at least one taxoid. Such methods can be practiced in vivo or in vitro, and can be used to add oxygen atoms to various intermediates in the Taxol® biosynthetic pathway, as well as to add oxygen atoms to related taxoids that are not necessarily on a Taxol® biosynthetic pathway. These methods include for example, adding oxygen atoms to acylation or glycosylation variants of paclitaxel, baccatin III, or 10-deacetyl-baccatin III. Such variants include, cephalomannine, xylosyl paclitaxel, 10-deactyl paclitaxel, paclitaxel C, 7-xylosyl baccatin III, 2-debenzoyl baccatin III, 7-xylosyl 10-baccatin III and 2-debenzoyl 10-baccatin III.

Additionally, the reduced form of any one of the disclosed oxygenases can be contacted with carbon monoxide and the carbon monoxide/oxygenase complex and be detected.

Sequence Listings

The nucleic acid and amino acid sequences listed in the accompanying sequence listing are shown using standard letter abbreviations for nucleotide bases, and three-letter code for amino acids. Only one strand of each nucleic acid sequence is shown, but the complementary strand is understood to be included by any reference to the displayed strand.

SEQ ID NOS: 1-21 are the nucleic acid sequences of the twenty-one different respective amplicons generated from the mRNA-reverse transcription-PCR.

SEQ ID NOS: 22-42 are the deduced amino acid sequences of the nucleic acid sequences shown in SEQ ID NOS: 1-21, respectively.

SEQ ID NOS: 43-55 are the full-length nucleic acid sequences of thirteen respective oxygenases.

SEQ ID NOS: 56-68 are the deduced amino acid sequences of the nucleic acid sequences shown in SEQ ID NOS: 43-55, respectively.

SEQ ID NOS: 69-72 are the PCR primers used in the RACE protocol.

SEQ ID NOS: 73-80 are PCR primers used to amplify the twenty-one different amplicons.

SEQ ID NOS: 81-86 are the full-length nucleic acid sequences of six respective oxygenases.

SEQ ID NOS: 87-92 are the full-length amino acid sequences of six respective oxygenases corresponding to the nucleic acid sequences shown in SEQ ID NOS: 81-90, respectively.

SEQ ID NOS: 93 and 94 are PCR primers that were used to clone oxygenases into FastBac-I vector (Life Technologies).

### BRIEF DESCRIPTION OF THE DRAWINGS

FIG. 1 shows an outline of early steps of the Taxol® biosynthetic pathway illustrating cyclization of geranylgeranyl diphosphate to taxadiene by taxadiene synthase (a), hydroxylation and rearrangement of the parent olefin to taxadien-5α-ol by taxadiene 5α-hydroxylase (b), acetylation by taxadienol-O-acetyl transferase (c), and hydroxylation to taxadien-5α-acetoxy-10β-ol by the taxane 10β-hydroxylase (d). The broken arrow indicates several as yet undefined steps.

FIG. 2 shows the proposed sequence for the hydroxylation of taxa-4(5),11(12)-diene to the level of a pentaol based on the relative abundances of naturally occurring taxoids. The reactions are catalyzed by cytochrome P450 oxygenases.

FIG. 3 shows a possible mechanism for the construction of the oxetane ring of Taxol® from the 4(20)-ene-5α-acetoxy functional grouping. Cytochrome P450-catalyzed epoxidation of the 4(20)-double bond, followed by intramolecular acetate migration and oxirane ring opening, could furnish the oxetane moiety.

FIG. 4 shows P450-specific forward primers that were used for differential display of mRNA-reverse-transcription polymerase chain reaction (DD-RT-PCR). Eight nondegenerate primers were necessary to cover all possible nucleotide sequences coding for the proline, phenylalanine, glycine (PFG) motif. Anchors were designed by Clontech as components of the kit.

FIGS. 5A-5D show relationships between the full-length amino acid sequences of the isolated oxygenases. FIG. 5A is a dendrogram showing peptide sequence relationships between some published, related plant cytochrome P450s and those cloned from T. cuspidata. For the published sequences, the first four letters of each name are genus and species abbreviations, CYP is the abbreviation for cytochrome P450, the following two numbers indicate the P450 family, and any additional letters and numbers refer to the subfamily. Cloned sequences from T. cuspidata are denoted by “F” followed by a number. The genus and species abbreviations are as follows: Lius—Linum usitatissimum; Paar—Parthenium argentatum; Caro—Catharanthus roseus; Some—Solanum melongena; Arth—Arabidopsis thaliana; Hetu—Helianthus tuberosus; Ziel—Zinnia elegans; Poki—Populus kitamkensis; Glma—Glycine max; Phau—Phaseolus aureus; Glec—Glycyrrhiza echinata; Mesa—Medicago sativa; Pisa—Pisum sativum; Pecr—Petroselinum crispum; Zema—Zea mays; Nita—Nicotiana tabacum; Eugr—Eustoma grandiflorum; Getr—Gentiana triflora; Peam—Persea americana; Mepi—Mentha piperita; Thar—Thlaspi arvense; Best—Berberis stolonifera; Soly—Solanum lycopersicum; Sobi—Sorghum bicolor; Potr—Populus tremuloides; Soch—Solanum chacoense; Nera—Nepeta racemosa; Cama—Campanula medium; Pehy—Petunia hybrida. FIG. 5B shows a pairwise comparison of certain Taxus cytochrome P450 clones. FIG. 5C is a dendrogram showing the relationships between the full-length peptide sequences of the disclosed proteins. The dendrogram was created using the Clustral method. The sequence identity data used as the basis of the dendrogram was created using the Sequence Distance function of the Megalign program of the lasergene (Version 99) package from DNAStar™. FIG. 5D is a similarity/identity table. The sequence identity data was generated using the same program as that used for generating the dendrogram shown in FIG. 5C and the similarity data was generated using the Olddistance function of GCG™ (version GCG10).

FIGS. 6A-6E show a reversed-phase HPLC radio-trace illustrating the conversion of [20-³H₂]taxa-4(20),11(12)-dien-5α-ol to more polar products by yeast transformants expressing Taxus cuspidata P450 genes and mass spectrum results. FIG. 6A shows the HPLC radio-trace of the authentic substrate [20-³H₂]taxa-4(20),11(12)-dien-5α-ol. FIGS. 6B and 6C show the HPLC radio-trace of the substrate [20-³H₂]taxa-4(20),11(12)-dien-5α-ol (26.33 min) and more polar products (retention ˜15 min) obtained after incubation with yeast transformed with clones F12 (SEQ ID NO: 43) and F9 (SEQ ID NO: 48), respectively. FIGS. 6D and 6E show the mass spectrum of the products (at 15.76 minutes and at 15.32 minutes, respectively) formed during the incubation of taxadien-5α-ol with yeast transformants expressing clones F12 and F9, respectively. Cytochrome P450 clones F14 (SEQ ID NO: 51) and F51 (SEQ. ID NO: 47) behaved similarly in yielding diol products.

FIG. 7 shows a 500 MHz proton NMR spectrum of the taxadien-diol monoacetate in benzene-d₆.

FIG. 8 shows a ¹H detected two-dimensional heteronuclear single quantum coherence (HSQC) NMR spectrum of the unknown taxadien-diol monoacetate.

FIGS. 9A and 9B show a ¹H—¹H two-dimensional homonuclear rotating frame NMR of the diol monoacetate. FIG. 9A is a total correlation spectrum (TOSCY) and FIG. 9B is a rotating frame n.O.e. (ROESY).

FIGS. 10A-10E show slices from the TOCSY spectrum taken along the F2, directly detected, axis.

FIGS. 11A-11E show slices from the ROESY spectrum taken along the F2, directly detected, axis.

FIG. 12 is a scheme for early hydroxylation steps in taxoid biosynthesis. The product of the first cytochrome P450-mediated oxygenation step, 5α-hydroxy taxadiene, can be either hydroxylated to 5α, 13α-dihydroxy taxadiene, or acetylated prior to hydroxylation at the C10β- and C14β-positions. Subsequent hydroxylation steps in the formation of Taxol® are undefined.

FIG. 13 is a graph illustrating reversed-phase radio-HPLC analysis of the biosynthetic product (Rₜ=26.3 min) generated from exogenous 5α-acetoxy-10β-hydroxy taxadiene (Rₜ=36.1 min) administered to yeast cells harboring cytochrome P450 cDNA clone F72. The product purified by this means was identified by GC-MS and NMR methods as 5α-acetoxy-10β-hydroxy taxadiene.

FIG. 14 is a graph illustrating reversed-phase radio-HPLC analysis of the biosynthetic product (Rₜ=36.3 min) generated from exogenous 5α-acetoxy taxadiene (Rₜ=51.9 min) administered to yeast cells harboring cytochrome P450 cDNA clone F72. The product purified by this means was identified by GC-MS and NMR methods as 5α-acetoxy-14β-hydroxy taxadiene.

FIG. 15 is a graph illustrating GC-MS analysis of the purified biosynthetic product (Rₜ=15.34 min) generated from exogenous 5α-acetoxy-10β-hydroxy taxadiene administered to yeast cells harboring cytochrome P450 cDNA clone F72.

FIG. 16 is a graph illustrating GC-MS analysis of the purified biosynthetic product (Rₜ=14.23 min) generated from exogenous 50α-acetoxy taxadiene administered to yeast cells harboring cytochrome P450 cDNA clone F72.

FIG. 17 illustrates an alignment of the deduced amino acid sequence alignment of the Taxus cuspidata taxoid-14β-hydroxylase (T10H.pro). Black boxes indicate identical residues for all three sequences; shaded boxes indicate identical residues for two of the sequences.

### DETAILED DESCRIPTION

Explanations

Host cell: A “host cell” is any cell that is capable of being transformed with a recombinant nucleic acid sequence. For example, bacterial cells, fungal cells, plant cells, insect cells, avian cells, mammalian cells, and amphibian cells. A host cell can be independent or can exist as a part of a transgenic organism (microorganism or macroorganism).

Taxoid: A “taxoid” is a chemical based on the Taxane ring structure as described in Kingston et al., Progress in the Chemistry of Organic Natural Products, Springer-Verlag, 1993.

Isolated: An “isolated” biological component (such as a nucleic acid or protein or organelle) is a component that has been substantially separated or purified away from other biological components in the cell of the organism in which the component naturally occurs, i.e., other chromosomal and extra-chromosomal DNA, RNA, proteins, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids.

Orthologs: An “ortholog” is a gene encoding a protein that displays a function similar to a gene derived from a different species.

Homologs: “Homologs” are multiple nucleotide sequences that share a common ancestral sequence and that diverged when a species carrying that ancestral sequence split into at least two species.

Purified: The term “purified” does not require absolute purity; rather, it is intended as a relative term. Thus, for example, a purified enzyme or nucleic acid preparation is one in which the subject protein or nucleotide, respectively, is at a higher concentration than the protein or nucleotide would be in its natural environment within an organism. For example, a preparation of an enzyme can be considered as purified if the enzyme content in the preparation represents at least 50% of the total protein content of the preparation.

Vector: A “vector” is a nucleic acid molecule as introduced into a host cell, thereby producing a transformed host cell. A vector may include nucleic acid sequences, such as an origin of replication, that permit the vector to replicate in a host cell. A vector may also include one or more screenable markers, selectable markers, or reporter genes and other genetic elements known in the art.

Transformed: A “transformed” cell is a cell into which a nucleic acid molecule has been introduced by molecular biology techniques. As used herein, the term “transformation” encompasses all techniques by which a nucleic acid molecule might be introduced into such a cell, including transfection with a viral vector, transformation with a plasmid vector, and introduction of naked DNA by electroporation, lipofection, and particle-gun acceleration.

DNA construct: The term “DNA construct” is intended to indicate any nucleic acid molecule of cDNA, genomic DNA, synthetic DNA, or RNA origin. The term “construct” is intended to indicate a nucleic acid segment that may be single- or double-stranded, and that may be based on a complete or partial naturally occurring nucleotide sequence encoding one or more of the oxygenase genes of the present invention. It is understood that such nucleotide sequences include intentionally manipulated nucleotide sequences, e.g., subjected to site-directed mutagenesis, and sequences that are degenerate as a result of the genetic code. All degenerate nucleotide sequences are included within the scope of the invention so long as the oxygenase encoded by the nucleotide sequence maintains oxygenase activity as described below.

Recombinant: A “recombinant” nucleic acid is one having a sequence that is not naturally occurring in the organism in which it is expressed, or has a sequence made by an artificial combination of two otherwise-separated, shorter sequences. This artificial combination is accomplished often by chemical synthesis or, more commonly, by the artificial manipulation of isolated segments of nucleic acids, e.g., by genetic engineering techniques. “Recombinant” also is used to describe nucleic acid molecules that have been artificially manipulated, but contain the same control sequences and coding regions that are found in the organism from which the gene was isolated.

Specific binding agent: A “specific binding agent” is an agent that is capable of specifically binding to the oxygenases of the present invention, and may include polyclonal antibodies, monoclonal antibodies (including humanized monoclonal antibodies) and fragments of monoclonal antibodies such as Fab, F(ab′)₂, and Fv fragments, as well as any other agent capable of specifically binding to the epitopes on the proteins.

cDNA (complementary DNA): A “cDNA” is a piece of DNA lacking internal, non-coding segments (introns) and regulatory sequences that determine transcription. cDNA is synthesized in the laboratory by reverse transcription from messenger RNA extracted from cells.

ORF (open reading frame): An “ORF” is a series of nucleotide triplets (codons) coding for amino acids without any termination codons. These sequences are usually translatable into respective polypeptides.

Operably linked: A first nucleic acid sequence is “operably linked” with a second nucleic acid sequence whenever the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Probes and primers: Nucleic acid probes and primers may readily be prepared based on the amino acid sequences and nucleic acid sequences provided by this invention. A “probe” comprises an isolated nucleic acid attached to a detectable label or reporter molecule. Typical labels include radioactive isotopes, ligands, chemiluminescent agents, and enzymes. Probes are typically shorter in length than the sequences from which they are derived (i.e., cDNA or gene sequences). For example, the amplicons shown in SEQ ID NOS: 1-21 and fragments thereof can be used as probes. One of ordinary skill in the art will appreciate that probe specificity increases with the length of the probe. For example, a probe can contain less than 800 bp, 700 bp, 600 bp, 500 bp, 400 bp, 300 bp, 200 bp, 100 bp, or 50 bp of constitutive bases of any of the oxygenase encoding sequences disclosed herein. Methods for labeling and guidance in the choice of labels appropriate for various purposes are discussed, e.g., in Sambrook et al. (eds.), Molecular Cloning. A Laboratory Manual, 2nd ed., vols. 1-3, Cold Spring Harbor Laboratory Press, Cold Spring Harbor, N.Y., 1989, and Ausubel et al. (eds.), Current Protocols in Molecular Biology, Greene Publishing and Wiley-Interscience, New York (with periodic updates), 1987.

“Primers” are short nucleic acids, preferably DNA oligonucleotides 10 nucleotides or more in length. A primer may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR), or other nucleic-acid amplification methods known in the art.

Methods for preparing and using probes and primers are described, for example, in references such as Sambrook et al. (eds.), Molecular Cloning. A Laboratory Manual, 2nd ed., vols. 1-3, Cold Spring Harbor Laboratory Press, Cold Spring Harbor, N.Y., 1989; Ausubel et al. (eds.), Current Protocols in Molecular Biology, Greene Publishing and Wiley-Interscience, New York (with periodic updates), 1987; and Innis et al., PCR Protocols: A Guide to Methods and Applications, Academic Press: San Diego, 1990. PCR primer pairs can be derived from a known sequence, for example, by using computer programs intended for that purpose such as Primer (Version 0.5, ©1991, Whitehead Institute for Biomedical Research, Cambridge, Mass.). One of skill in the art will appreciate that the specificity of a particular probe or primer increases with the length of the probe or primer. Thus, for example, a primer comprising 20 consecutive nucleotides will anneal to a target with higher specificity than a corresponding primer of only 15 nucleotides in length. Thus, in order to obtain greater specificity, probes and primers may be selected that comprise, for example, 10, 20, 25, 30, 35, 40, 50 or more consecutive nucleotides.

Sequence identity: The similarity between two nucleic acid sequences or between two amino acid sequences is expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene 73:237-244, 1988; Higgins & Sharp, CABIOS 5:151-153, 1989; Corpet et al., Nucleic Acids Research 16:10881-10890, 1988; Huang, et al., Computer Applications in the Biosciences 8:155-165, 1992; and Pearson et al., Methods in Molecular Biology 24:307-331, 1994. Altschul et al., J. Mol. Biol. 215:403-410, 1990, presents a detailed consideration of sequence-alignment methods and homology calculations.

The National Center for Biotechnology Information (NCBI) Basic Local Alignment Search Tool (BLAST™, Altschul et al. J. Mol. Biol. 215:403-410, 1990) is available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

For comparisons of amino acid sequences of greater than about 30 amino acids, the “Blast 2 sequences” function of the BLAST™ program is employed using the default BLOSUM62 matrix set to default parameters (gap existence cost of 11, and a per residue gap cost of 1). When aligning short peptides (fewer than approximately 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 45%, at least 50%, at least 60%, at least 80%, at least 85%, at least 90%, or at least 95% sequence identity.

As mentioned above, “sequence identity” can be determined by using an alignment algorithm such as Blast™ (available at the National Center for Biotechnology Information [NCBI]). A first nucleic acid is “substantially similar” to a second nucleic acid if, when optimally aligned (using the default parameters provided at the NCBI wesite) with the other nucleic acid (or its complementary strand), there is nucleotide sequence identity in at least about, for example, 50%, 75%, 80%, 85%, 90% or 95% of the nucleotide bases. Sequence similarity can be determined by comparing the nucleotide sequences of two nucleic acids using the BLAST™ sequence analysis software (blastn) available from NCBI. Such comparisons may be made using the software set to default settings (expect=10, filter=default, descriptions=500 pairwise, alignments=500, alignment view=standard, gap existence cost=11, per residue existence=1, per residue gap cost=0.85). Similarly, a first polypeptide is substantially similar to a second polypeptide if they show sequence identity of at least about 75%-90% or greater when optimally aligned and compared using BLAST software (blastp) using default settings.

Oxygenase activity: Enzymes exhibiting oxygenase activity are capable of directly incorporating oxygen into a substrate molecule. Oxygenases can be either dioxygenases, in which case the oxygenase incorporates two oxygen atoms into the substrate; or, monooxygenases, in which only one oxygen atom is incorporated into the primary substrate to form a hydroxyl or epoxide group. Thus, monooxygenases can be referred to as “hydroxylases.” Taxoid oxygenases are a subset of oxygenases that specifically utilize taxoids as substrates.

Oxygenases: Oxygenases are enzymes that display oxygenase activity as described supra. However, all oxygenases do not recognize the same substrates. Therefore, oxygenase enzyme-activity assays may utilize different substrates depending on the specificity of the particular oxygenase enzyme. One of ordinary skill in the art will appreciate that the spectrophotometry-based assay described below is a representative example of a general oxygenase activity assay, and that direct assays can be used to test oxygenase catalysis directed towards different substrates.

II. Characterization of Oxygenases

A. Overview of Experimental Procedures

Biochemical studies have indicated that at least the first five oxygenation steps of the Taxol® pathway are catalyzed by cytochrome P450 hydroxylases (the remaining three oxygenations also are likely catalyzed by cytochrome P450 enzymes). These studies also have shown that these are slow steps of the reaction pathway and, thus, important candidates for cDNA isolation for the purpose of over-expression in relevant producing organisms to increase Taxol® yields (Croteau et al., Curr. Topics Plant Physiol. 15:94-104, 1995; and Hezari et al., Planta Med. 63:291-295, 1997). Protein purification of cytochrome P450 enzymes from Taxus microsomes (Hefner et al., Methods Enzymol. 272:243-250, 1996), as a basis for cDNA cloning, was not performed because the number of P450 species present, and their known similarity in physical properties (Mihaliak et al., Methods Plant Biochem. 9:261-279, 1993), would almost certainly have prevented bringing the individual proteins to homogeneity for amino acid microsequencing.

Therefore, a strategy based on the differential display of mRNA-reverse transcription-PCR (DD-RT-PCR) was used for isolating transcriptionally active cytochrome P450s in Taxus cells, which previous biochemical studies had shown to undergo substantial up-regulation of the Taxol® pathway 16 hours after induction with methyl jasmonate (Hefner et al., Arch. Biochem. Biophys. 360:62-74, 1998). Differential display experimental schemes allow for the identification of mRNA species that are up-regulated in response to certain stimulus. Generally, one set of samples is not treated with the stimulant, and a second set of samples is treated with the stimulant. Subsequently, the mRNA from both groups is isolated and amplified. The mRNA of interest is identified by comparing the mRNA from the stimulated and unstimulated samples. The mRNA that is present only in the stimulated sample appears to represent genes that are activated upon stimulation.

In the experiments described below, mRNA from an untreated cell culture was compared to the mRNA from a culture that had been induced with methyl jasmonate for 16 hours. In order to obtain predominantly induced cytochrome P450 sequences, forward primers were designed based on a conserved proline, phenylalanine, glycine (PFG) motif in plant cytochrome P450 genes. The use of primers directed toward the (PFG) motif in conjunction with the DD-RT-PCR-based strategy revealed roughly 100 differentially expressed species, and the sequences of 100 of these were obtained and analyzed. Of these, 39 represented PCR products containing a cytochrome P450-type sequence. Analysis of these sequences revealed that the C-terminus from 21 different and unique cytochrome P450 genes had been isolated. The 21 nucleic acid sequences amplified (amplicons) and identified as regions encoding oxygenases are shown in SEQ ID NOS: 1-21, respectively.

Twelve amplicons were labeled and used as hybridization probes to screen the methyl jasmonate-induced T. cuspidata cell cDNA library. Screening the T. cuspidata library allowed identification of nine full-length clones. Four additional clones, which were truncated at the 5′-terminus, were obtained in full-length form using a 5′-RACE (Rapid analysis of cDNA ends) method to acquire the missing 5′-sequences. Thus, the initial use of the amplicons, described above, has allowed for the identification of thirteen full-length oxygenases (SEQ ID NOS: 43-55, respectively). Subsequently, various molecular techniques were used to identify an additional 10 full-length cDNAs (SEQ ID NOS: 81-86, respectively) and their corresponding amino acid sequences (SEQ ID NOS: 87-92, respectively).

The full-length oxygenase clones identified through the use of the amplicon-based probes can be cloned into prokaryotic-based and eukaryotic-based expression systems. Once expressed, the functional competence of the resulting oxygenases can be assessed using the spectrophotometric assay described below.

The clones that are found to be active using the spectrophotometric assay are at a minimum useful for detecting carbon monoxide. Additionally, in the examples provided below, several of the full-length oxygenase-encoding sequences are shown to have in situ oxygenase activity toward taxoids when expressed in Saccharomyces cerevisiae and baculovirus-Spodoptera cells.

Oxygenases produced by cloned full-length oxygenase-encoding sequences also can be tested for the ability to oxygenate taxoid substrates in vivo. This can be done by feeding taxoid intermediates to transgenic cells expressing the cloned oxygenase-encoding sequences.

B. Cloning of Oxygenases

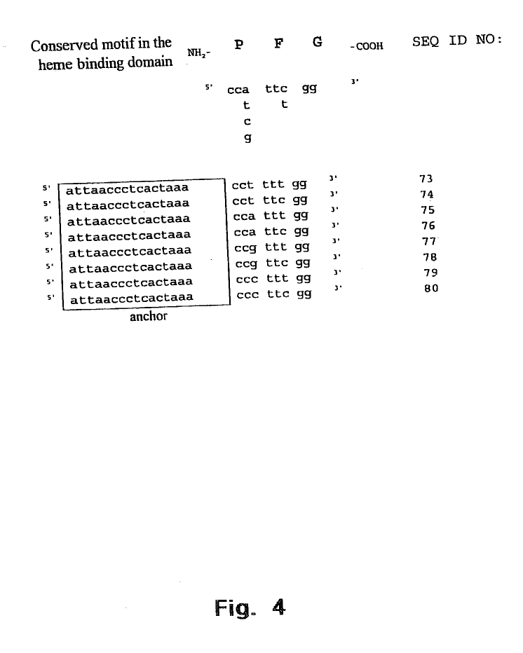

As described supra, a DD-RT-PCR scheme was used for the isolation of transcriptionally active cytochrome P450s in Taxus cells, which previously had been shown to undergo substantial up-regulation of the Taxol® pathway 16 hours after induction with methyl jasmonate (Hefner et al., Arch. Biochem. Biophys. 360×62-74, 1998). Because an increase in the relevant enzyme activities resulted from induction (indicating de novo protein synthesis), mRNA from an untreated cell culture was compared to mRNA from a culture that had been so induced for 16 hours. In order to obtain predominantly induced cytochrome P450 sequences, forward primers were designed based on a conserved motif in plant cytochrome P450 genes. Related strategies have been used with other plants (Schopfer and Ebel, Mol. Gen. Genet. 258×315-322, 1998). The proline, phenylalanine, glycine (PFG) motif is a well-conserved region of the heme-binding domain (Durst and Nelson, “Diversity and Evolution of Plant P450 and P450 Reductase,” in Durst and O'Keefe (eds.), Drug Metabolism and Drug Interactions, Freund, UK, 1995, pp. 189-206). The corresponding codons of this region contain only two degenerate positions; thus, a set of only eight non-degenerate primers was necessary to encompass all sequence possibilities (FIG. 4). This PFG motif is located 200-250 bp upstream of the stop codon, and the length of the 3′-untranslated region should range between 100 and 300 bp. Thus, the length of the expected PCR fragments would be in the 300-550 bp range. This DD-RT-PCR-based strategy revealed roughly 100 differentially expressed species, and the sequences of 100 of these were obtained and analyzed. Of these, 39 represented PCR products containing a cytochrome P450-type sequence. Analysis of these sequences revealed that the C-terminus from 21 different and unique cytochrome P450 genes had been isolated. These DNA fragments (12 thus far) are being used as labeled hybridization probes to screen the methyl jasmonate-induced T. cuspidata cell cDNA library. By this means, nine clones have been obtained in full-length form by screening. Four additional clones, which were truncated at the 5′-terminus, were obtained in full-length form using a 5′-RACE (Rapid analysis of cDNA ends) method to acquire the missing 5′-sequences.

C. Sequence Analysis

The full-length oxygenase sequences initially obtained (using 12 partial sequence probes) were compared pairwise. It was shown that a total of 13 unique sequences (showing less than 85% similarity), designated clones F12, F21, F42, F31, F51, F9, F56, F19, F14, F55, F34, F72, and F10, respectively (SEQ ID NOS: 43-55, respectively) were present. Two of the isolated clones, clone F51 (SEQ ID NO: 47) and clone F9 (SEQ ID NO: 48) were not identical to any of the 21 C-terminal fragments originally found by the DD-RT-PCR cloning strategy, bringing the total number of initially identified unique oxygenase genes, and gene fragments, to 23.

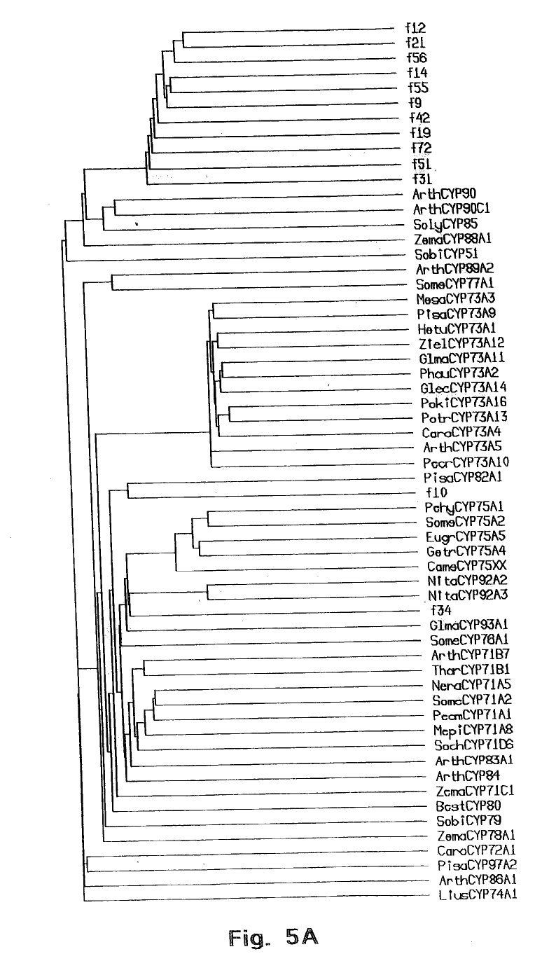

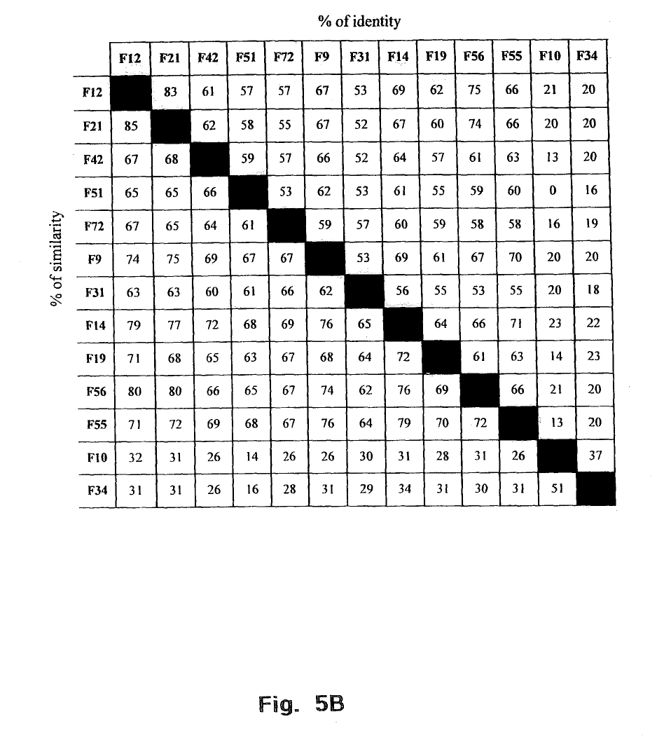

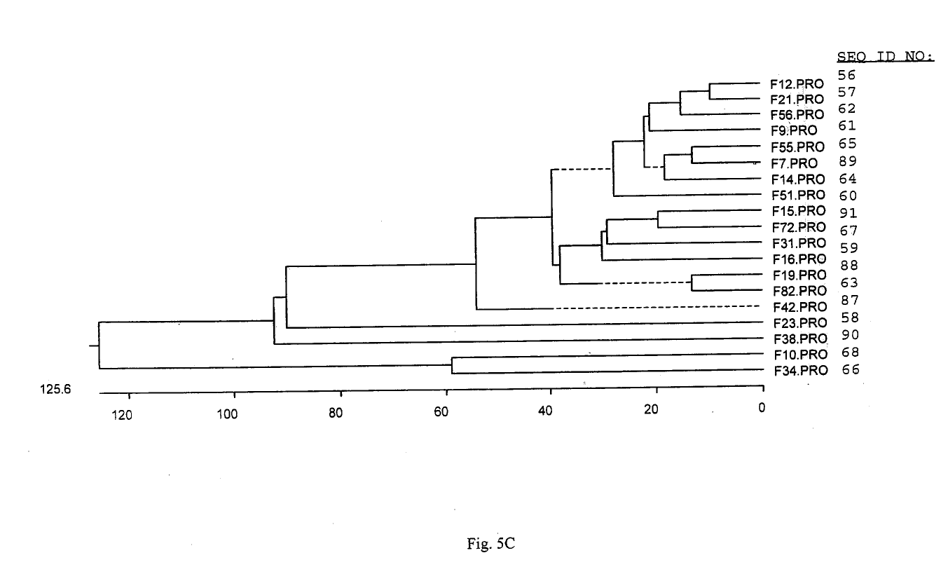

The clones obtained also were compared pairwise to all known plant cytochrome P450 oxygenase sequences in the databases (provided at the NCBI website) (FIGS. 5A and 5B) provide a dendrogram of these relationships and a table of pairwise similarity and identity comparisons).

This analysis revealed that 11 of the Taxus clones sorted into one cytochrome P450 family. This large group of related clones seems to resemble most closely the CYP90, CYP85, and CYP88 cytochrome P450 families. Some members of these families are known to be involved in terpenoid metabolism [e.g., gibberellin (diterpene, C20) and brassinosteroid (triterpene C30) biosynthesis], suggesting that the cytochrome P450 clones obtained from Taxus could be involved in the biosynthesis of the diterpenoid Taxol®. Table 1 lists accession numbers of relevant sequences and related information. Outlying clones F10 (SEQ ID NO: 55) and F34 (SEQ ID NO: 53) are related more closely to CYP family 82 (phenylpropanoid metabolism) and CYP family 92 (unknown function), respectively.

After the initial 13 full-length clones were identified, six more were isolated. Thus, the total number of full-length oxygenase clones identified is nineteen. A dendrogram showing the relationship of all of the identified oxygenase clones is provided in FIG. 5C. A table providing both the sequence identity and similarity of the clones is provided in FIG. 5D.

TABLE 1
Closest Relatives to Taxus Cytochrome P450 Sequences

| | | Clones That Are |
|---|---|---|
| Family | Description | Similar |
| CYP90A1 | Arabidopsis thaliana GenEMBL X87367 | F9, F12, F14, F19, F21, |
| | mRNA (1608 bp); GenEMBL X87368 gene | F31, F42, F51, F55, F56, |
| | (4937 bp). Szekeres et al., “Brassinosteroids | and F72 |
| | rescue the deficiency of CYP90, a | (SEQ ID NOS: 48, 43, |
| | cytochrome P450, controlling cell elongation | 51, 50, 44, 46, 45, 47, |
| | and de-etiolation in Arabidopsis,” Cell | 52, 49, and 54, |
| | 85:171-182 (1996). | respectively) |
| CYP85 | Solanum lycopersicum (tomato) (also | F9, F12, F14, F19, F21, |
| | Lycopersicon esculentum) GenEMBL | F31, F42, F51, F55, F56, |
| | U54770 (1395 bp). Bishop et al., “The | and F72 (SEQ ID NOS: |
| | tomato dwarf gene isolated by heterologous | 48, 43, 51, 50, 44, 46, |
| | transposon tagging encodes the first member | 45, 47, 52, 49, and 54, |
| | of a new family of cytochrome P450,” Plant | respectively) |
| | Cell 8:959-969 (1996). | |
| CYP88A1 | Zea mays GenEMBL U32579 (1724 bp). | F9, F12, F14, F19, F21, |
| | Winkler and Helentjaris, “The maize dwarf3 | F31, F42, F51, F55, F56, |
| | gene encodes a cytochrome P450-mediated | and F72 (SEQ ID NOS: |
| | early step in gibberellin biosynthesis,” Plant | 48, 43, 51, 50, 44, 46, |
| | Cell 7:1307-1317 (1995). | 45, 47, 52, 49, and 54, |
| | | respectively) |
| CYP82A1 | Pisum sativum (pea) GenEMBL U29333 | Outlying Clone F10 |
| | (1763 bp). Frank et al., “Cloning of | (SEQ ID NO: 55) |
| | phenylpropanoid pathway P450 | |
| | monooxygenases expressed in Pisum | |
| | sativum,” unpublished. | |
| CYP82A2 | Glycine max (soybean) GenEMBL Y10491 | Outlying Clone F34 |
| | (1757 bp). Schopfer and Ebel, | (SEQ ID NO: 53) |
| | “Identification of elicitor-induced | |
| | cytochrome P450s of soybean (Glycine max | |
| | L.) using differential display of mRNA,” | |
| | Mol. Gen. Genet. 258:315-322 (1998). | |
| CYP92A2 | Nicotiana tabacum (tobacco) GenEMBL | Outlying Clone F34 |
| | X95342 (1628 bp). Czernic et al., | (SEQ ID NO: 53) |
| | “Characterization of hsr201 and hsr215, two | |
| | tobacco genes preferentially expressed | |
| | during the hypersensitive reaction provoked | |
| | by phytopathogenic bacteria,” unpublished. | |

D. Functional Expression

Functional cytochrome P450 expression can be obtained by using the pYeDP60 plasmid in yeast (Saccharomyces cerevisiae) engineered to co-express one or the other of a cytochrome P450 reductase from Arabidopsis thaliana; the plant-derived reductase is important for efficient electron transfer to the cytochrome (Pompon et al., Methods Enzymol. 272:51-64, 1999).,

Since a functional P450 cytochrome, in the appropriately reduced form, will bind competently to carbon monoxide and give a characteristic CO-difference spectrum (Omura and Sato, J. Biol. Chem. 239:2370-2378, 1964), a spectrophotometric means for assessing, and quantitatively estimating, the presence of functional recombinant cytochrome P450 in transformed yeast cells by in situ (in vivo) measurement was developed. Of the 19 full-length cytochrome P450 clones from Taxus thus far obtained, ten have yielded detectable CO-difference spectra (Table 2). It is expected that cytochrome P450 clones that do not yield reliable expression in S. cerevisiae can be transferred to, expressed in, and confirmed by CO-difference spectrum utilizing alternative prokaryotic and eukaryotic systems. These alternative expression systems for cytochrome P450 genes include the yeast Pichia pastoris, for which expression vectors and hosts are commercially available (Invitrogen, Carlsbad, Calif.), as well as established E. coli and baculovirus-insect cell systems for which general expression procedures have been described (Barnes, Methods Enzymol. 272:1-14, 1996; Gonzalez et al., Methods Enzymol. 206:93-99, 1991; Lee et al., Methods Enzymol. 272:86-98, 1996; and Lupien et al., Arch. Biochem. Biophys. 368:181-192, 1999).

Clones that prove to be capable of binding to CO are useful at least for detecting CO in various samples. Additional cases are described in the Examples below. Further testing of the recombinantly expressed clones may prove that they are additionally useful for adding one or more oxygen atoms to taxoid substrates.

E. In vivo Assays of Yeast Cells Expressing Recombinant Oxygenases

1. Use of Substrates [20-³H₃]taxa-4(5),11(12)-diene or [20-³H₂]taxa-4(20,11(12)-dien-5α-ol

Transformed yeast cells that functionally express a recombinant cytochrome P450 gene from Taxus (by CO-difference spectrum) can be tested in vivo for their ability to oxygenate (hydroxylate or epoxidize) taxoid substrates fed exogenously to the cells, thereby eliminating the need for microsome isolation for preliminary in vitro assays.

Accordingly, several clones of the available full-length clones were expressed in induced yeast host cells. These cells were fed [20-³H₃]taxa-4(5),11(12)-diene or [20-³H₂]taxa-4(20,11(12)-dien-5α-ol in separate incubations and compared to untransformed controls similarly fed (and that were shown to be inactive with taxoid substrates). The extracts resulting from these incubations were analyzed by radio-HPLC, and the clones that yielded a product are shown below in Table 2.

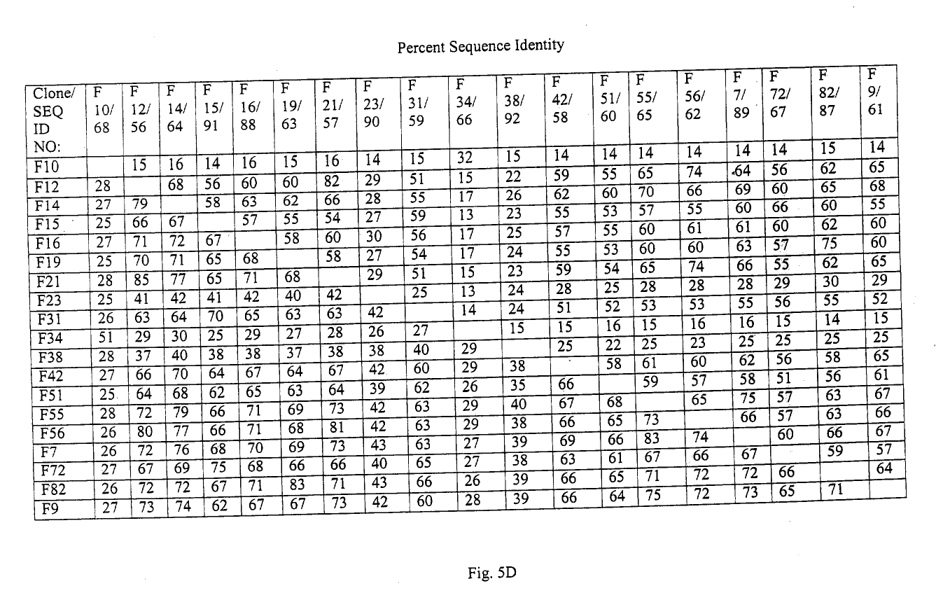

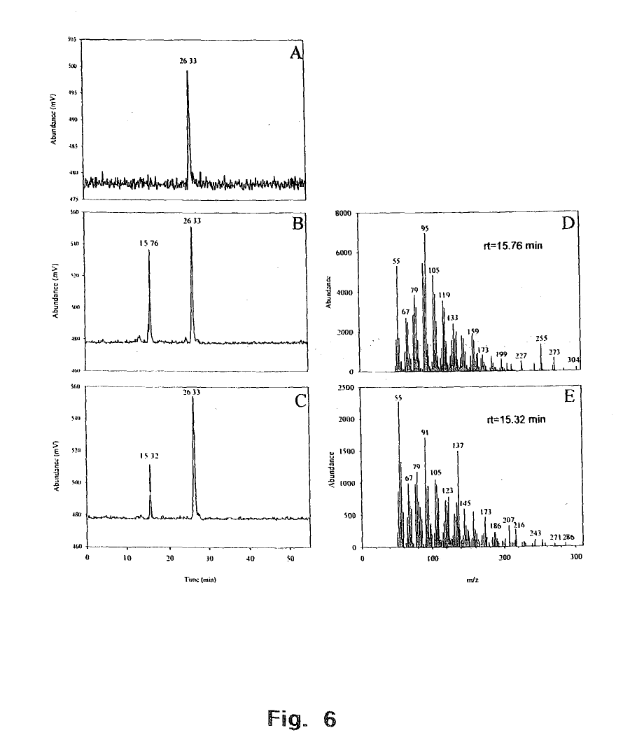

Representative HPLC traces are shown in FIGS. 6A-6C. Representative GC-MS (gas chromatography-mass spectrometry) analyses of the products from an incubation are shown in FIGS. 6D and 6E. The results shown in FIGS. 6A-6E confirm that two distinctly different taxadien-diols derived from taxadien-5α-ol were formed, one yielding the expected parent ion at P⁺=m/z 304, and the other less stable to the conditions of the analysis in losing water readily to yield the highest mass ion at m/z 286 (P⁺—H₂O).

2. Use of Substrate [20-³H₂] taxa-4(20), 11(12)-dien-5α-yl acetate

Transformed yeast cells that functionally express a recombinant cytochrome P450 gene from Taxus (by CO-difference spectrum) were tested in vivo for their ability to oxygenate (hydroxylate or epoxidize) taxoid substrates fed exogenously, thereby eliminating the need for microsome isolation for such a preliminary in vitro assay. The clones indicated in Table 2, below, were induced in yeast host cells that were fed [20-³H₂]taxa-4(20),11(12)-dien-5α-yl acetate in separate incubations and compared to untransformed controls similarly fed (and that were shown to be inactive with taxoid substrates). The ether extracts resulting from these incubations were analyzed by radio-HPLC. Several clones converted the taxadienyl-5α-yl acetate substrate to a more polar product.

TABLE 2

| Full-length | Probe name | CO | | product |
|---|---|---|---|---|
| name (SEQ | (SEQ ID NO: | diff. | | identified |
| ID NO: nt/aa) | nt/aa) | spec. | assayed with | HPLC peak |
| F12 | aa1 | + | Taxadiene | No |
| *43/56 | 11/32 | | Taxadien5αol | ++ |
| | | | Taxadienyl | ++ |
| | | | Ac | |
| F21 | cb1 | + | Taxadiene | No |
| *44/57 | 10/31 | | Taxadien5αol | + |
| | | | Taxadienyl | No |
| | | | Ac | |
| F31 | ab2 | + | Taxadiene | No |
| *46/59 | 1/22 | | Taxadien5αol | No |
| | | | Taxadienyl | No |
| | | | Ac | |
| F42 | ai2 | − | Taxadiene | No |
| *45/58 | 5/26 | | Taxadien5αol | No |
| F51 | Lib. Screen | + | Taxadiene | No |
| *47/60 | | | Taxadien5αol | ++ |
| | | | Taxadienyl | ++ |
| | | | Ac | |
| F72 | cm2 | + | Taxadiene | No |
| *54/67 | 19/40 | | Taxadien5αol | + |
| | | | Taxadienyl | + |
| | | | Ac | |
| F82 | dl1 | − | Taxadiene | No |
| 81/87 | 20/41 | | Taxadien5αol | + |
| | | | Taxadienyl | ++ |
| | | | Ac | |
| F9 | Lib. Screen | + | Taxadiene | No |
| *48/61 | | | Taxadien5αol | + |
| | | | Taxadienyl | +/− |
| | | | Ac | |
| F56 | el2 | − | Taxadiene | No |
| *49/62 | 8/29 | | Taxadien5αol | No |
| F14 | ea1 | +++ | Taxadiene | No |
| *51/64 | 13/34 | | Taxadien5αol | ++ |
| | | | Taxadienyl | ++ |
| | | | Ac | |
| F19 | ds1 | − | Taxadiene | No |
| *50/63 | 14/35 | | Taxadien5αol | No |
| F55 | cf2 | − | Taxadiene | No |
| *52/65 | 6/27 | | Taxadien5αol | No |
| F16 | ae1 | +++ | Taxadiene | No |
| 82/88 | 2/23 | | Taxadien5αol | ++ |
| | | | Taxadienyl | ++ |
| | | | Ac | |
| F7 | cj1 | − | Taxadiene | No |
| 83/89 | 7/28 | | Taxadien5αol | ++ |
| | | | Taxadienyl | ++ |
| | | | Ac | |
| F23 | di1 | − | Taxadiene | No |
| 84/90 | 15/36 | | Taxadien5αol | No |
| F10 | ba1 | + | Taxadiene | No |
| *55/68 | 17/38 | | Taxadien5αol | No |
| F34 | du1 | ++ | Taxadiene | No |
| *53/66 | | | Taxadien5αol | No |
| F15 | df | | | |
| 85/91 | 12/33 | | | |
| F38 | ad6 | | | |
| 86/92 | 16/37 | | | |

Additional testing of the metabolite of clone F14 (SEQ ID NO: 64) was conducted. The metabolite isolated by HPLC was subjected to GC-MS analysis and shown to possess a retention time (compared to the starting material) and mass spectrum that were consistent with respective data obtained from a taxadien-diol monoacetate. The parent ion (P⁺) was observed at m/z 346 (taxadienyl acetate (MW=330) plus O) with diagnostic ions at m/z 328 (P⁺—H₂O), 313 (P⁺—H₂O—CH₃), 286 (P⁺—CH₃COOH), 271 (P⁺—CH₃COOH—CH₃), 268 (P⁺—CH₃COOH—H₂O) and 253 (P⁺—CH₃COOH—CH₃—H₂O).

Preparative-scale incubations of the transformed yeast harboring clone F14 (SEQ ID NO: 51), with the taxadien-5α-yl acetate substrate, yielded the HPLC-based isolation of about 100 μg of the unknown diol monoacetate (>97% purity by GC) for NMR analysis. Since all of the ¹H resonances of taxadien-4(20),11(12)-dien-5α-ol (and of the acetate ester) had been assigned previously (Hefner et al., Chem. and Biol. 3:479-489, 1996), elucidation of the structure of the unknown diol monoacetate was accomplished by ¹H detection experiments (sample-size-limited direct ¹³C measurements).

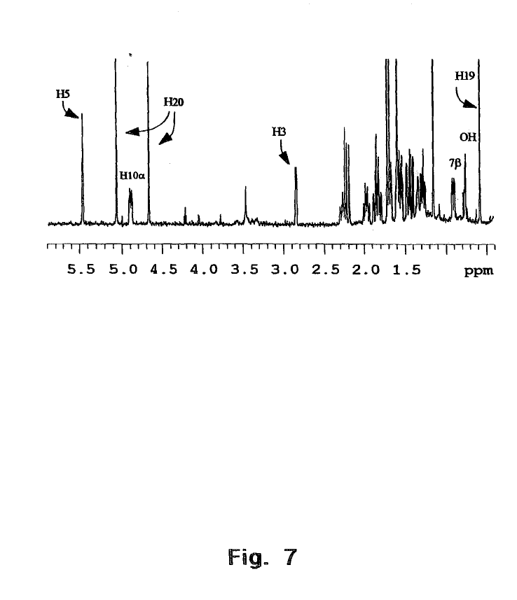

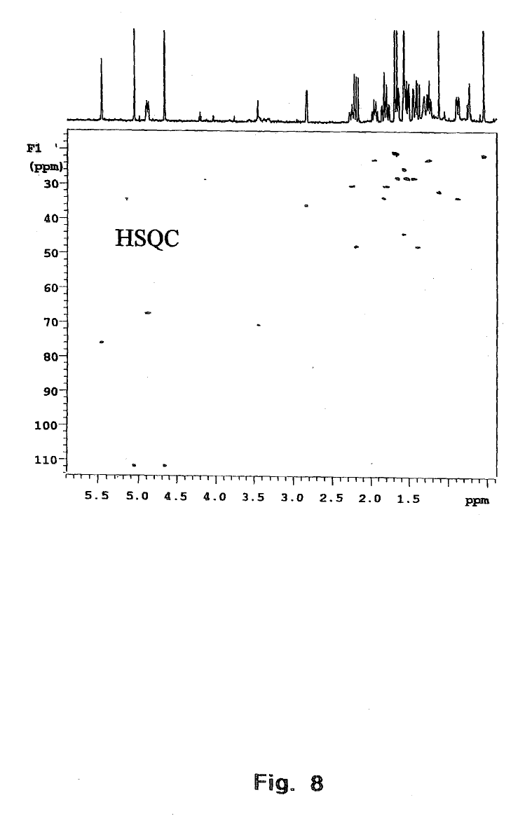

The ¹H-NMR spectrum is illustrated in FIG. 7, and Table 3, below, lists the complete ¹H assignments along with their respective one-carbon correlated ¹³C assignments as determined indirectly from heteronuclear single quantum coherence (HSQC; FIG. 8). The assignments are consistent with those of other known taxadien monool and diol derivatives. For example, chemical shifts for C5 δ75.9, C5; δ5.47, H5) and C10 (δ67.2, C10; δ4.9 H10) are assigned as oxy-methines. The shifts for C20 (δ111.6, C20; δ5.07, H20, exo; δ4.67, H20, endo) are consistent with the exocyclic methylene observed in other taxa-4(20),11(12)-dienes. Other characteristic shifts are observed for H7α (δ1.84), H19 methyl (δ0.56), H3 (δ2.84), and the gem-dimethyls H16 (δ1.14, exo) and H17 (δ1.59, endo).

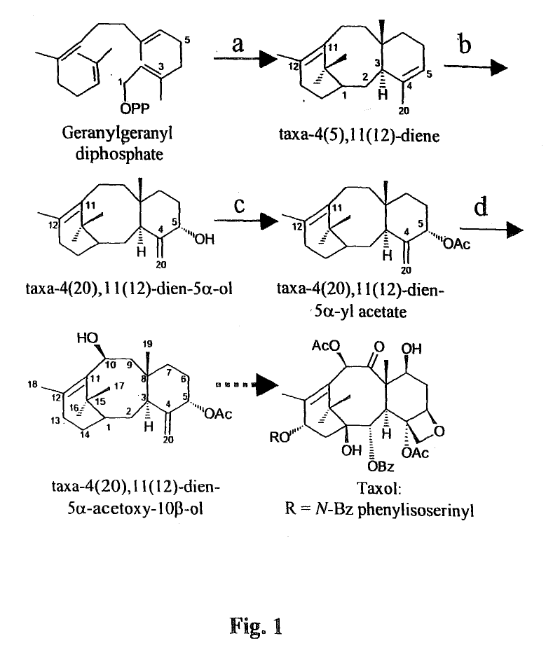

TABLE 3
Complete ¹H-NMR assignments and one-bond
correlated ¹³C assignments (as measured
indirectly from HSQC) for the biosynthetic product
derived from taxadien-5α-yl
acetate by the cytochrome P450 expressed from clone
F14. For position numbering, see FIG. 1.

| Position | Carbon | α-proton | β-proton |
|---|---|---|---|
| number | (δ) | (δ) | (δ) |
| 1 | 43.9 | | 1.59 |
| 2 | 28 | 1.47 | 1.53 |
| 3 | 35.9 | 2.84 | |
| 4 | | | |
| 5 | 75.9 | | 5.47 |
| 6 | 27.9 | 1.66 | 1.55 |
| 7 | 33.6 | 1.94 | 0.9 |
| 8 | | | |
| 9 | 47.6 | 1.42 | 2.21 |
| 10 | 67.2 | 4.9 | |
| 11 | | | |
| 12 | | | |
| 13 | 30.3 | 1.8 | 2.26 |
| 14 | 22.7 | 1.26 | 1.96 |
| 15 | | | |
| 16 | 31.8 | 1.14 (exo) | |
| 17 | 25.3 | 1.59 (endo) | |
| 18 | 20.7 | 1.71 | |
| 19 | 21.4 | | 0.66 |
| 20 | 111.6 | 5.07 (exo) | |
| | | 4.67 (endo) | |
| 21 (acetate) | 21 | 1.66 | |

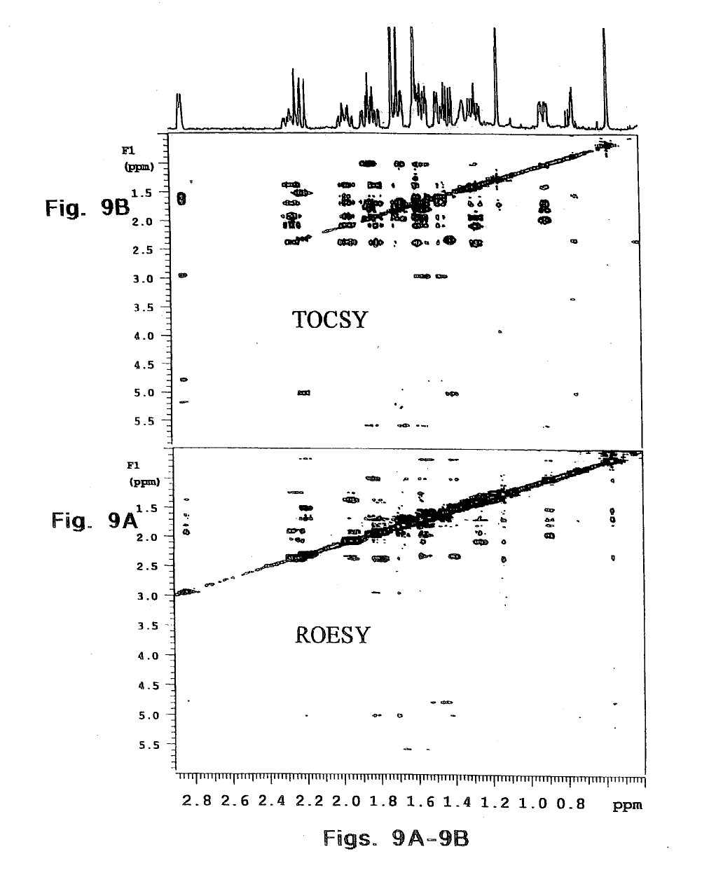

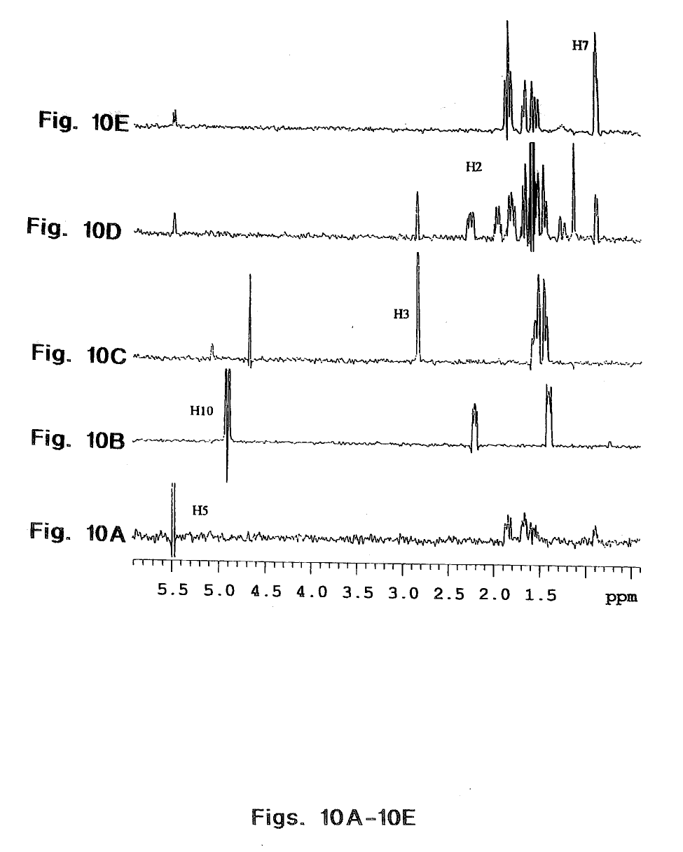

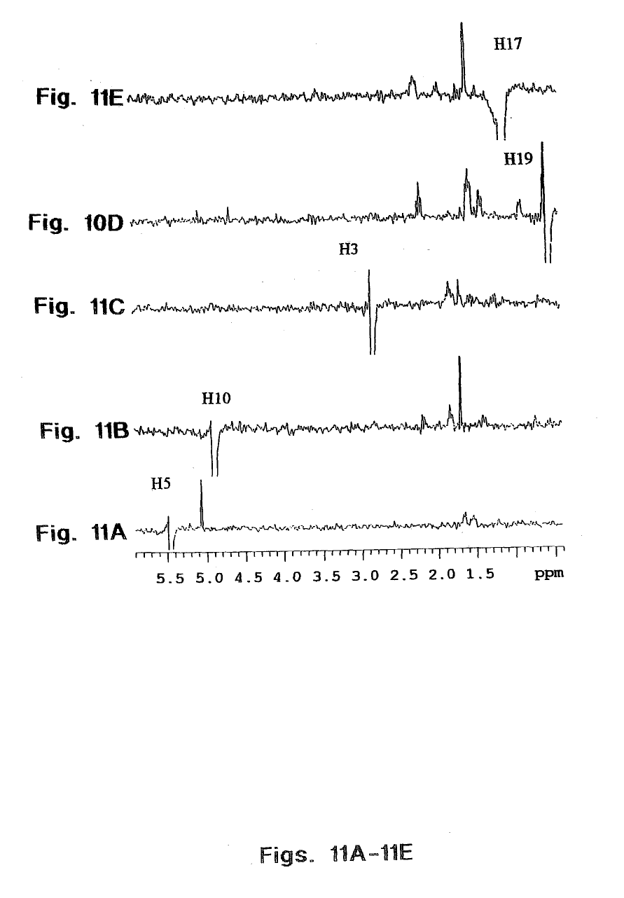

The 2D-TOCSY spectra (FIGS. 9A and 10) complemented the HSQC data and permitted additional regiochemical assignments. The H5 proton (δ5.47) (FIGS. 10A and 10E) was correlated strongly with H6 (δ1.66, 1.55) and H7 (δ1.94, δ0.9) protons but had no appreciable coupling to either of the H20 signals (δ5.07, δ4.67) or to H3 (δ2.84), which is a common feature observed with taxadiene derivatives. The spin system defined in part by H3 (δ2.84), H2 (δ1.47 and δ1.53), H1 (δ1.59), H13 (δ1.80, δ2.26), and H14 (δ1.26, δ1.96) was apparent in FIGS. 10C and 10E. The H18 allylic methyl (δ1.71) also displayed a weak correlation with H13. In contrast to the extended spin correlations noted in FIG. 10D, the H9 (δ1.42, δ2.21) and H10 (δ4.9) signals formed an isolated spin system (see FIG. 10B), which included the H10 hydroxyl (δ0.85). A correlation also was observed between the two gem-dimethyl signals (δ1.14 and δ1.59), which was consistent with the spectra of other taxadiene derivatives.

¹H-¹H ROESY (Rotational nuclear Overhauser Effect Spectroscop Y) is useful for determining the particular signals that arise from protons situated closely in space but are not closely connected by chemical bonds. Therefore, 2D-ROESY spectra (FIGS. 9B and 11) were used to confirm the regiochemical assignments and to assess relative stereochemistry (Several of these n.O.e correlations are listed in Table 4). As used herein, “n.O.e.” refers to the nuclear Overhauser effect and can also be abbreviated as “NOE.” The nuclear Overhause effect (abbreviated “n.O.e.” or “NOE”) is a secondary effect of decoupling of nuclei that causes changes in peak areas of an NMR spectrum. The maximum value of this increase is governed by the relative magnitudes of the magnetogyric ratios for the decoupled and observed nuclides, which can be expressed mathematically as NOE (max)=1-γ_{dec}/γ_{obs}.

¹H-¹H TOCSY (TOtal Correlated Spectroscop Y) is useful for determining the particular signals that arise from protons within a spin system, especially whenever the multiplets overlap or there is extensive second-order coupling. The 2D-TOCSY (total correlation spectrum), described herein, showed that a second heteroatom was introduced into the C9-C10 fragment, but the regiochemistry was ambiguous based on this single measurement. The 2D-ROESY confirmed that oxidation had occurred at C10 and placed the C10 hydroxyl in the β-orientation. This assignment also was supported by an observed n.O.e between the H10 proton (δ4.90) (FIG. 11B) and the allylic methyl, H18 (δ1.71), which is consistent with an α-configuration for H10. Additional stereochemical assignments were made by noting correlations between H9β (δ2.21) and the H17 methyl which must be endo (δ1.59) (FIG. 11E), the H19 methyl (δ0.56) which is β-oriented, and the H2β-proton (δ1.53). The other H9 signal (δ1.42) correlated with H19 and the H7β-proton (δ0.90), as well as H10 (δ4.90) (FIGS. 11D and 11B). It also was noted that ³J_{HH} was large (11.7 Hz) between the H9β- and H10α-protons, consistent with a nearly axial arrangement for this pair; a smaller coupling (5.3 Hz) between H9α and H10 was consistent with an equatorial configuration between these two protons.

ROESY spectroscopy also was used to confirm the stereochemistry at H5. Moderately strong correlations were seen between H5 (δ5.47) (see Table 4 and FIG. 11A) and both C6 signals (δ1.66, δ1.55), consistent with an equatorial orientation for H5. The ³J_{HH} coupling was quite small (<3 Hz) between H5 and all other scalar-coupled partners, providing further evidence for the adopted equatorial orientation of H5. A moderately strong n.O.e between H5 and H20exo was noted, but there were no n.O.e correlations observed between H5 and other protons on the α-face of the molecule. These results confirmed that H5 was β-configured and that the acetate group was α-oriented as in the substrate. One other significant structural motif in taxadiene derivatives was the near occlusion of the H3 proton on the α-face due to the unusual folding of the molecule, thereby making the H3 proton (δ2.84) a useful probe for this face. Indeed, n.O.e correlations were observed between H3, H10, H13α, and the allylic methyl H18 (Table 4, below, and FIG. 11C).

TABLE 4
n.O.e. Correlations

| Proton | | n.0.e. | correlations | | | | |
|---|---|---|---|---|---|---|---|
| H3 | alpha | 10 (w) | 13-a (m) | 18 (w) | | | |
| H5 | beta | 20-exo (m) | 6-ab (m) | | | | |
| H7 | beta | 19 (w) | 9-a (m) | 6-ab (m) | 7-a (s) | | |
| H7 | alpha | 7-b (s) | 3 (m) | 10 (m) | 21 (w) ? | | |
| H9 | alpha | 9-b (s) | 7-b (m-w) | 19 (w) | 9-a (m) | OH | |
| | | | | | | (w) | |
| H9 | beta | 17 (m) | 9-a (s) | 2-b (w) | 19 (w) | | |
| H10 | alpha | 7-a (m) | 18 (m) | 9-a (m) | 19-b (w) | OH | |
| | | | | | | (w) | |
| H13 | beta | 14-b (m) | 13-a (s) | 18 (vw) | 16-exo (m) | | |
| H14 | alpha | 3 (w) | 14-b (s) | 13-a (m) | | | |
| H14 | beta | 14-a (s) | 16-exo (m) | 1 (m) | 13-b (m) | | |
| H16 | exo | 17-endo (m) | 3-b (m) | 14-b (m-w) | 1 (w) | | |
| H19 | beta | 20-endo (w) | 20-exo (w) | 7-b (m) | 9-ab (m) | 2-b | 6-b |
| | | | | | | (s) | (m) |
| H20 | endo | 20-exo (s) | 3 (w) | 2-a (s) | 19 (w) | | |
| H20 | exo | 20-endo (m) | 5 (m) | | | | |

This full assignment of the structure confirms the identity of the biosynthetic product as taxa-4(5),11(12)-dien-5α-acetoxy-10β-ol, and indicates that a cDNA encoding the cytochrome P450 taxane 10β-hydroxylase has been isolated. This 1494-bp cDNA (SEQ ID NO: 5 1) translates a 497-residue deduced protein having a molecular weight of 56,690 Daltons and that bears a typical N-terminal membrane anchor (Brown et al., J. Biol. Chem. 264:4442-4449, 1989), a hydrophobic insertion segment (Nelson et al., J. Biol. Chem. 263:6038-6050, 1988), and a stop-transfer signal (Sakaguchi et al., EMBO J. 6:2425-2431, 1987). The protein possesses all of the conserved motifs anticipated for cytochrome P450 oxygenases, including the oxygen-binding domain (Shimada et al., in Bunabiki (ed.), Oxygenases and Model Systems, Kluwer, Boston, Mass., pp. 195-221, 1997), the highly conserved heme-binding motif (Durst et al., Drug Metab. Drug Interact. 12:189-206, 1995; and von Wachenfeldt et al., in Ortiz de Montellano (ed.), Cytochrome P450: Structure, Mechanism, and Biochemistry, Plenum, New York, N.Y., pp. 183-223, 1995), and the PFG element (amino acids 435-437).

F. In Vitro Assays of Isolated Enzymes for Taxoid Oxygenase Activity

The standard enzyme assay for assessing oxygenase activity of the recombinant cytochrome P450 employed the following conditions: 25 mM HEPES buffer, pH 7.5, 400 μM NADPH, 300 μg protein and 30 μM substrate (taxadiene, taxadienol, or taxadienyl acetate) in a total volume of 1 mL. Samples were incubated at 32° C. for 12 hours, after which 1 mL of saturated NaCl solution was added to the reaction mixture, followed by extraction of the product with 2 mL of hexane/ethyl acetate (4:1, v/v). The extracts were dried and dissolved in acetonitrile for product analysis by radio-HPLC [column: Alltech Econosil C18 5 μm particle size (250 mm×4.6 mm): solvent system A: 0.01% (v/v) H₃PO₄, 2% acetonitrile, 97.99% H₂O; solvent system B: 0.01% H₃PO₄, 99.99 acetonitrile; gradient: 0-5 minutes, 100% A; 5-15 minutes, 0-50% B; 15-55 minutes, 50-100% B; 55-65 minutes, 100% B; 65-70 minutes, 0-100% A; 70-75 minutes, 100% A; flow rate 1 mL/minute; for detection, a radio-chromatography detector (Flow-One®-Beta Series A-100, Radiomatic) was used].

Of the three test substrates (A, B, C), taxadiene was not converted detectably to an oxygenated product by recombinant cytochrome P450 clone F16 (SEQ ID NO: 93). Of the 5α-ol derivatives, taxa-4(20),11(12)-dien-5α-ol was converted most efficiently to a diol product as determined by GC-MS analysis (parent ion indicating a molecular weight of 304). Preparative incubations with taxadienol allowed the generation of ˜100 μg of the diol product that was purified by a combination of reversed phase HPLC, as described above, and normal phase TLC (silica gel with toluene/acetone (3:1, v/v)) in preparation for structural determination by ¹H- and ¹³C-NMR analysis (500 MHz). Comparison of spectra to those of authentic taxa-4(20),11(12)-dien-5α-ol (Hefner et al., Chem. Biol. 3:479-489, 1996) indicated that the product of the clone F16 (SEQ ID NO: 93) cytochrome P450 oxygenase reaction is taxa-4(20),11(12)-dien-5u.,13cc-diol. These results indicated that clone F16 (SEQ ID NO: 16) encodes a cytochrome P450 taxane 13α-hydroxylase, likely representing a later hydroxylation step of the Taxol® biosynthetic pathway.

Additionally, biochemical studies can be done to determine the particular diol that resides on the Taxol® pathway (i.e., the gene encoding the next pathway step suspected to be responsible for C10 hydroxylation), and to determine the particular activities (and genes) that reside further down the pathway (catalyzing formation of triol, tetraol, pentaol, etc.) but that yield a cytochrome P450 oxygenase capable of catalyzing the hydroxylation of taxadien-5α-ol as an adventitious substrate. Other expression systems also can be tested to obtain functional expression of the remaining clones, and all functional clones are being tested with other taxoid substrates.

It is notable that some of the clones that are capable of transforming taxoid intermediates are from the same, closely related family (see placement of clones F9, F12, F14, and F51 (SEQ ID NOS: 61, 56, 64, and 60) in the dendrogram of FIG. 5(A)). Outlying clone 34, although it yielded a reliable CO-difference spectrum (confirming a functional cytochrome P450 and its utility for detecting CO), does not transform the taxoid substrates to oxygenated products. However, this clone, when expressed in a different expression system, may prove to be active against other taxoid substrates.

III. Other Oxygenases of the Taxol® Pathway

The protocol described above yielded twenty-one related amplicons. Initial use of twelve amplicons as probes for screening the cDNA library allowed for the isolation and characterization of thirteen oxygenase-encoding DNA sequences. Subsequently, additional full-length enzymes were isolated. Several of these full-length sequences were expressed recombinantly and tested in situ, and ten were shown to be capable of binding CO, and, therefore, to be useful for detecting CO (Table 2). Additionally, nine clones were shown to be capable of hydroxylating taxoid substrates in vivo (Table 2).

There are at least five distinct oxygenases in the Taxol® biosynthetic pathway (Hezari et al., Planta Med. 63:291-295, 1997), and the close relationship between the nucleic acid sequences of the 21 amplicons indicates that the remaining amplicon sequences represent partial nucleic acid sequences of the other oxygenases in the Taxol® biosynthetic pathway. Hence, the above-described protocol enables the identification and recombinant production of oxygenases corresponding to the full-length versions of the 21 amplicon sequences provided. Therefore, the following discussion relating to Taxol® oxygenases refers to the full-length oxygenases shown in the respective sequence listings, as well as the remaining oxygenases of the Taxol® biosynthetic pathway that are identifiable through the use of the amplicon sequences. Furthermore, the remaining oxygenases can be tested for enzymatic activity using “functional assays,” such as the spectrophotometric assay described below and direct assays for catalysis with the appropriate taxoid substrates.

IV. Isolating Oxygenases of the Taxol® Biosynthetic Pathway

A. Cell Culture

Initiation, propagation, and induction of Taxus species cell cultures have been previously described (Hefner et al., Arch. Biochem. Biophys. 360:62-75, 1998). Enzymes and reagents were obtained from United States Biochemical Corp. (Cleveland, Ohio), Gibco BRL (Grand Island, N.Y.), Promega (Madison, Wis.), and New England BioLabs, Inc. (Beverly, Mass.), and were used according to the manufacturers' instructions. Chemicals were purchased from Sigma Chemical Co. (St. Louis, Mo.).

B. Vectors and DNA Manipulation

Unless otherwise stated, all routine DNA manipulations and cloning were performed by standard methods (Sambrook et al. (eds.), Molecular Cloning. A Laboratory Manual, 2nd ed., vols. 1-3, Cold Spring Harbor Laboratory Press, Cold Spring Harbor, N.Y., 1989). PCR amplifications were performed by established procedures (Innis, et al., PCR Protocols. A Guide to Methods and Applications, Academic Press, New York, 1990). DNA was sequenced using Amplitaq™ DNA polymerase (Roche, Somerville, N.J.) and fluorescence cycle sequencing on an Applied Biosystems Prism™ 373 DNA Sequencer (Perkin-Elmer, Norwalk, Conn.). The Saccharomyces cerevisiae expression vector pYeDP60 was as described previously (Pompon et al., Methods Enzymol. 272:51-64, 1996).

C. E. coli and Yeast Strains

The E. coli strains XLI-Blue MRF′ (Stratagene, La Jolla, Calif.) and TOP10F′ (Invitrogen, Carlsbad, Calif.), were used for routine cloning and for cloning PCR products, respectively. The yeast strains used for expression each expressed one of two different Arabidopsis thaliana cytochrome P450 reductases, and were designated WAT11 and WAT21, respectively (Pompon et al., Methods Enzymol. 272:51-64, 1996).

D. cDNA Library Construction

A cDNA library was prepared from mRNA isolated from T. cuspidata suspension cell cultures, which had been induced to maximal Taxol® production with methyl jasmonate for 16 hours. Isolation of total RNA from 1.5 g T. cuspidata cells was developed empirically using a buffer containing 4 M guanidine thiocyanate, 25 mM EDTA, 14 mM 2-mercaptoethanol, and 100 mM Tris-HCl, pH 7.5. Cells were homogenized on ice using a polytron (VWR Scientific, Salt Lake City, Utah) (4×15-second bursts at setting 7). The homogenate was adjusted to 2% (v/v) Triton X-100 and allowed to stand 15 minutes on ice, after which an equal volume of 3 M sodium acetate, pH 6.0 was added. After mixing, the solution was incubated on ice for an additional 15 minutes, followed by centrifugation at 15,000 g for 30 minutes at 4° C. The supernatant was mixed with 0.8 volume of isopropanol and left to stand on ice for 5 minutes. After centrifugation at 15,000 g for 30 minutes at 4° C., the resulting pellet was redissolved in 8 mL 20 mM Tris-HCl, pH 8.0, containing 1 mM EDTA, then adjusted to pH 7.0 by addition of 2 mL 2 M NaCl in 250 mM MOPS buffer at pH 7.0. Total RNA was recovered by passing this solution over a nucleic-acid-isolation column (Qiagen, Valencia, Calif.) following the manufacturer's instructions. Poly(A)⁺ RNA was purified by using the Oligotex™ mRNA kit following the manufacturer's instructions (Qiagen, Valencia, Calif.). Messenger RNA prepared in this fashion was used to construct a library using a λZAPII™-cDNA synthesis kit and ZAP-cDNA Gigapack III™ gold packaging kit (Stratagene, La Jolla, Calif.) following the manufacturer's instructions. The isolated mRNA also was used to construct a RACE (Rapid Amplification of cDNA Ends) library using a Marathon cDNA amplification kit (Clontech, Palo Alto, Calif.).

E. Differential Display of mRNA

Differential display of mRNA was performed using the Delta Differential Display Kit (Clontech, Palo Alto, Calif.) by following the manufacturer's instructions except were noted. Total RNA was isolated as described above from two different Taxus cuspidata suspension cell cultures, one that had been induced with methyl jasmonate 16 hours before RNA isolation and the other that had not been treated (i.e., uninduced). Cytochrome P450-specific forward primers (FIG. 4), instead of random primers, were used in combination with reverse-anchor-(dT)9N−1N−1 primers (where N−1=A, G, or C) provided in the kit. The anchor designed by Clontech was added to each P450-specific primer to increase the annealing temperature after the fourth low-stringency PCR cycle; this led to a significant reduction of the background signal. Each cytochrome P450-specific primer was used with the three anchored oligo(dT) primers terminated by each nucleotide. PCR reactions were performed with a RoboCycler™ 96 Temperature Cycler (Stratagene, La Jolla, Calif.), using one cycle at 94° C. for 5 minutes, 40° C. for 5 minutes, 68° C. for 5 minutes, followed by three cycles at 94° C. for 30 seconds, 40° C. for 30 seconds, 68° C. for 5 minutes, and 32 cycles at 94° C. for 20 seconds, 60° C. for 30 seconds, and 68° C. for 2 minutes. Finally, the reactions were heated at 68° C. for 7 minutes. The resulting amplicons were separated on a 6% denaturing polyacrylamide gel (HR-100, Genomyx Corporation, Foster City, Calif.) using the LR DNA Sequencer Electrophoresis System (Genomyx Corporation).

Differential display bands of interest were cut from the dried gel, eluted with 100 mL of 10 mM Tris-HCl buffer, pH 8.0, containing 1 mM EDTA, by incubation overnight at 4° C. A 5-mL, aliquot of the extract was used to re-amplify the cDNA fragment by PCR using the same primers as in the original amplification. The reactions initially were heated to 94° C. for 2 minutes, then subjected to 30 cycles at 94° C. for 1 minute, 60° C. for 1 minute, and 68° C. for 2 minutes. Finally, to facilitate cloning of the PCR product, the reactions were heated at 68° C. for 7 minutes. Amplicons were analyzed by agarose gel electrophoresis as before. Bands were excised from the gel and the DNA was extracted from the agarose. This gel-purified cDNA was then transferred into the T/A cloning vector pCR2.1-TOPO (Invitrogen, Carlsbad, Calif.).

The DD-RT-PCR-based screening revealed about 100 clearly differentially expressed bands, all of which were sequenced and analyzed. Of these, 39 represented PCR products containing cytochrome P450-like sequences. The nucleotide and deduced peptide sequences of these 39 amplicons were compiled using the GCG-fragment assembly programs and the sequence-alignment program “Pileup” (Genetics Computer Group, Program Manual for the Wisconsin Package, Version 9, Genetics Computer Group, 575 Science Drive, Madison, Wis., 1994). This comparison of cloned sequences revealed that C-terminal fragments from 21 different cytochrome P450 genes had been isolated. These cytochrome P450 sequences were used to prepare hybridization probes in order to isolate the corresponding full-length clones by screening the cDNA library.

F. cDNA Library Screening

Initially, twelve probes (SEQ ID NOS: 11, 10, 1, 5, 4, 19, 8, 17, 13, 14, 21, and 6, respectively) were labeled randomly using the Ready-To-Go™ kit (Amersham Pharmacia Biotech, Piscataway, N.J.) following the manufacturer's instructions. Plaque lifts of the T. cuspidata phage library were made on nylon membranes and were screened using a mixture of two radiolabeled probes. Phage DNA was cross-linked to the nylon membranes by autoclaving on fast cycle for 3 minutes at 120° C. After cooling, the membranes were washed for 5 minutes in 2×SSC (sodium citrate buffer). Prehybridization was performed for 1 to 2 hours at 65° C. in 6×SSC, containing 0.5% SDS, and 5× Denhardt's reagent. Hybridization was performed in the same buffer for 20 hours at 65° C. The nylon membranes were washed twice for 5 minutes each in 2×SSC with 0.1% SDS at room temperature, and twice for 1 hour each in 1×SSC with 0.1% SDS at 65° C. After washing, the membranes were exposed for 17 hours onto Kodak (Rochester, N.Y.) XAR™ film at −70° C. Positive plaques were purified through one additional round of hybridization. Purified λZAPII clones were excised in vivo as pBluescript II SK(+) phagemids (Stratagene, La Jolla, Calif.) and transformed into E. coli SOLR cells. The size of each cDNA insert was determined by PCR using T3 and T7 promoter primers. Inserts (>1.6 kb; of a size necessary to encode a typical cytochrome P450 of 50-60 kDa) were sequenced and sorted into groups based on sequence similarity/identity using the GCG fragment assembly programs (Genetics Computer Group, Program Manual for the Wisconsin Package, Version 9, Genetics Computer Group, 575 Science Drive, Madison, Wis., 1994). Each unique sequence was used as a query in database searching using either BLAST or FASTA programs (Genetics Computer Group, Program Manual for the Wisconsin Package, Version 9, Genetics Computer Group, 575 Science Drive, Madison, Wis., 1994), to define sequences with significant homology to plant cytochrome P450 sequences. These clones also were compared pairwise at both the nucleic acid and amino acid levels using the “Pileup” and “Gap” programs (Genetics Computer Group, Program Manual for the Wisconsin Package, Version 9, Genetics Computer Group, 575 Science Drive, Madison, Wis., 1994).

G. Generation of Full-Length Clones by 5′-RACE

Of the thirteen clones initially examined, full-length sequences of nine were obtained by screening of the T. cuspidata λ-phage library with the corresponding probes (clones F12, F21, F31, F42, F51, F72, F9, F56, and F10, respectively (SEQ ID NOS: 43, 44, 46, 45, 47, 54, 48, 49, and 55, respectively)). To obtain the 5′-sequence portions of the other four truncated clones F14, F19, F34, and F55 (SEQ ID NOS: 51, 50, 53 and 52, respectively), 5′-RACE was performed using the Marathon cDNA amplification kit (Clontech, Palo Alto, Calif.) according to the manufacturer's instructions. The reverse primers used were: for F14, 5′-TCGGTGATTGTAACGGAAGAGC-3′ (SEQ ID NO: 69); for F19, 5′-CTGGCTTTTCCAACGGAGCAT-GAG-3′ (SEQ ID NO: 70); for F34, 5′-ATTGTTTCTCAGCCCGCGCAGTATG-3′ (SEQ ID NO: 71); for F55, 5′-TCGGT-TTCTATGACGGAAGAGATG-3′ (SEQ ID NO: 72). Using the defined 5′-sequences thus acquired, and the previously obtained 3′-sequence information, primers corresponding to these terminal regions were designed and the full-length versions of each clone were obtained by amplification with Pfu polymerase (Stratagene, La Jolla, Calif.) using library cDNA as target. These primers also were designed to contain nucleotide sequences encoding restriction sites that were used to facilitate cloning into the yeast expression vector.

H. cDNA Expression of Cytochrome P450 Enzymes in Yeast

Appropriate restriction sites were introduced by standard PCR methods (Innis et al., PCR Protocols: A Guide to Methods and Applications, Academic Press, San Diego, Calif., 1990) immediately upstream of the ATG start codon and downstream of the stop codon of all full-length cytochrome P450 clones. These modified amplicons were gel-purified, digested with the corresponding restriction enzymes, and then ligated into the expression vector pYeDP60. The vector/insert junctions were sequenced to ensure that no errors had been introduced by the PCR construction. Verified clones were transformed into yeast using the lithium acetate method (Ito et al., J. Bacteriol. 153:163-168, 1983). Isolated transformants were grown to stationary phase in SGI medium (Pompon et al., Methods Enzymol. 272:51-64, 1996), and used as inocula for a large-scale expression culture grown in YPL medium (Pompon et al., Methods Enzymol. 272:51-64, 1996). Approximately 24 hours after induction of cytochrome P450 expression with galactose (to 10% final concentration), a portion of the yeast cell culture was harvested by centrifugation. One-half of the culture was treated with carbon monoxide, and the cytochrome P450 CO-difference spectrum was recorded directly (using untreated cells as a control) by spectrophotometry (Omura and Sato, J. Biol. Chem. 239:2370-2378, 1964).

This direct, in situ method for demonstrating the presence of functional, recombinant cytochrome P450, and for estimating the quantity of the competent enzyme, also can be applied to other expression systems, including E. coli, Pichia pastoris, insect cells (as described below), and Spodoptera fugiperda cells. Of the thirteen full-length clones obtained so far, eight exhibit a detectable CO-difference spectrum when the recombinant cytochrome P450 gene product is expressed in this yeast system and assayed by this in situ method.

I. cDNA Expression of Cytochrome P450 Enzymes in Insect Cells

As mentioned above, insect cell expression systems, such as the baculovirus-Spondoptera system described below, can be used to express the oxygenases described herein.

For example, the functional identification of the Taxus cuspidata cytochrome P450 clone F16 was accomplished using the baculovirus-Spodoptera expression system. (The use of this system for the heterologous expression of cytochrome P450 genes has been described previously (Asseffa et al., Arch. Biochem. Biophys. 274:481-490, 1989; Gonzalez et al., Methods Enzymol. 206:93-99, 1991; and Kraus et al., Proc. Natl. Acad. Sci. USA 92:2071-2075, 1995)). For the heterologous expression of clone F16 in Spodoptera fugiperda Sf9 cells with the Autographa californica baculovirus expression system, the F16 cytochrome P450 open reading frame (orf) was amplified by PCR using the F16-pYEDP60 construct as a template. For PCR, two gene-specific primers were designed that contained, for the purpose of subcloning the F16 orf into the FastBac-1 vector (Life Technologies), a BamHI and a NotI restriction site (forward primer 5′-gggatccATGGCCCTTAAGCAATTGGAAGTTTC-3′ (SEQ ID NO: 93); reverse primer 5′-ggcggccgcTTAAGATCTGGAATAGAGTTTAATGG-3′ (SEQ ID NO: 94)). The gel-purified PCR product so obtained was subcloned into the pCR-Blunt vector (Invitrogen, Carlsbad, Calif.). From the derived recombinant pCR-Blunt vector, the subcloned cytochrome P450 orf was excised using the added restriction sites, and the obtained DNA fragment was ligated into the BamHI/NotI-digested pFastBac1 vector (Life Technologies, Grand Island, N.Y.). The sequence and the correct insertion of clone F16 into the pFastBac1 vector were confirmed by sequencing of the insert. The pFastBac/F16orf construct was then used for the preparation of the recombinant Bacmid DNA by transformation of the Escherichia coli strain DH10Bac (Life Technologies). Construction of the recombinant Bacmid DNA and the transfection of Spodoptera frugiperda Sf9 cells were done according to the manufacturer's protocol.

The Spodoptera frugiperda Sf9 cell cultures were propagated either as adherent monolayer cultures in Grace insect cell culture medium (Life Technologies) supplemented with 10% FCS (Life Technologies) or as suspension cultures in Grace medium containing 10% FCS and 0.1% Pluronic F-68 (Sigman, St. Louis, Mo.). The adherent cell cultures were maintained in a chamber at 28° C. The suspension cultures were incubated in a shaker at 28° C. at 140 rpm. The adherent cell cultures were grown in T25 tissue culture flasks (Nalgene Nuc, Rochester, N.Y.) with passage of one-third to one-half of the culture every 2 to 3 days. For heterologous protein production, the cultures were grown as suspensions. The cells from two tissue culture flasks (80-90% confluent) were added to 50 mL of standard suspension insect culture medium in a 100-mL conical flask, and were incubated as above until a cell density of ˜2×10⁶ cells/mL was reached. The cells were collected by centrifugation at room temperature at 140 g for 10 minutes. The resulting cell pellet was resuspended in 1/10 of the original volume with fresh medium.

For the functional characterization of clone F16, the recombinant baculovirus carrying the cytochrome P450 clone F16 orf was coexpressed with a recombinant baculovirus carrying the Taxus NADPH:cytochrome P450 reductase gene. To the insect cell suspension, the two recombinant baculoviruses were added at a multiplicity of infection of 1-5. The viral titers were determined according to the End-Point Dilution method (O'Reilly et al., Baculovirus Expression Vectors, A Laboratory Manual, New York, N.Y., Freeman and Company, 1992). For infection, the cells were incubated for 1 hour at 28° C. and 80 rpm. The cell culture volume was brought to 50 mL with standard cell culture medium, and hemin (Sigma) was added to a final concentration of 2 μg/mL. The infected cells were incubated for 48 hours in a gyratory shaker at 28° C. and 140 rpm. The infected insect cells were harvested from the cell culture medium by centrifugation as described above, and washed twice with PBS (50 mM KH₂PO₄, pH 7.5, 0.9% NaCl). The cell pellet so obtained was resuspended in 5 mL of HEPES/DTT Buffer (25 mM HEPES, pH 7.5, 1 mM DTT). The cells were lysed by mild sonication (VirSonic, Virtis Company, Gardiner, N.Y.), the cell debris was removed by centrifugation at 5,000 g for 10 minutes at 4° C., and the resulting supernatant was collected for use in enzyme assays.

J. Assay of Recombinant Cytochrome P450 Activity Toward Taxoid Substrates

Isolated transformants for each full-length cytochrome P450 clone shown to express a functional enzyme by CO-difference spectrum (ten clones) were grown to stationary phase in 2 mL SGI medium at 30° C. and used to inoculate a 10-mL expression culture (in YPL medium). Approximately 8 hours after induction, cells were harvested by centrifugation (10 minutes at 1500 rpm), and the pellet was resuspended in 2 mL of fresh YPL medium.

To eliminate additional complication and uncertainty associated with microsome isolations for in vitro assays, 10⁶ dpm of [20-³H₃]taxa-4(5),11(12)-diene (16 Ci/mol) or [20-³H₂]taxa-4(20),11(12)-dien-5-α-ol (4.0 Ci/mol), or other taxoid substrate were added directly to the cell suspension to assay conversion in vivo. After 12 hours of incubation at 30° C. with agitation (250 rpm), the mixture was treated for 15 minutes in a sonication bath and extracted 3 times with 2 mL diethyl ether to insure isolation of the biosynthetic products. These ether extracts, containing residual substrate and derived product(s), were concentrated to dryness, resuspended in 200 μL of CH₃CN, and filtered. These samples were analyzed by radio-HPLC (Hefner et al., Chemistry and Biology 3:479-489, 1996) using a 4.6 mm i.d.×250 mm column of Econosil C18, 5μ (Alltech, Deerfield, Ill.) with a gradient of CH₃CN in H₂O from 0% to 85% (10 minutes at 1 mL/minute), then to 100% CH₃CN over 40 minutes.

The foregoing method is capable of separating taxoids ranging in polarity from taxadiene to approximately that of taxadien-hexaol. For confirmation of product type, gas chromatography-mass spectrometry (GC-MS) or liquid chromatography-mass spectrometry (LC-MS) is employed, depending on the volatility of the product.

In the present example, of the eight clones confirmed to be functional by CO difference spectra, four exhibited a hydroxylated product in situ when incubated with taxadien-5α-ol.

K. Substrate Preparation The syntheses of [20-³H₃]taxa-4(5),11(12)-diene (16 Ci/mol) and [20-³H₂]taxa-4(20),11(12)-dien-5α-ol (4.0 Ci/mol) have been described elsewhere (Hefner et al., Chemistry and Biology 3:479-489, 1996; and Rubenstein et al., J. Org. Chem. 60:7215-7223, 1995, respectively). Other taxane substrates (diols, triols, and tetraols of taxadiene) needed to monitor more advanced cytochrome P450-mediated bioconversions are generated by incubating radiolabeled taxa-4(20),11(12)-dien-5α-ol with isolated T. canadensis microsomes, or appropriate recombinant cytochrome P450 enzymes, and separating the products by preparative radio-HPLC. Taxusin (5α,9α,10β,13α-tetraacetoxy-taxa-4(20),11(12)-diene) is isolated from Taxus heartwood and purified by standard chromatographic procedures (De Case De Marcano et al., Chem. Commun. 1282-1294, 1969). Following deacetylation and reacetylation with [¹⁴C] acetic anhydride, this labeled substrate is used to monitor enzymatic hydroxylation at C1, C2, and C7 and epoxidation at C4-C20. 2α-Isobutyryloxy-5α, 7α, 10β-triaacetoxy-taxa-4(20),11(12)-diene, isolated from the same source (De Case De Marcano et al., Chem. Commun. 1282-1294, 1969), can be modified similarly to provide a substrate for monitoring hydroxylation at C9 and C13. If taxa-4(20),11(12)-dien-5α-ol is hydroxylated at C10 as an early step, then the surrogate substrates for examining enzymatic oxygenation at all relevant positions of the taxane ring can be procured.

L. NMR Spectrometry

All NMR spectra were recorded on a Varian Inova-500 NMR spectrometer operating at 18° C. using a very sensitive 5-mm pulsed-field-gradient ¹H indirect-detection probe. The taxadien-diol monoacetate was dissolved in C₆D₆ to a final concentration of about 300 μM. A 2D-TOCSY spectrum was acquired using a z-filtered DIPSI mixing sequence, a 60 msec mixing time, 10 kHz spin-lock field, 16 repetitions, 256 (t₁)×2048 (t₂) complex points, and 6500 Hz sweep in each dimension. The 2D-ROESY spectrum was acquired using a z-filtered mixing sequence with a 409-msec mixing time, 4-kHz spin-lock field, 128 repetitions, 256 (t₁)×2048 (t₂) complex points, and 6500-Hz sweep in each dimension. A 2D-HSQC spectrum was acquired using 256 repetitions, 128 (t₁)×1024 (t₂) complex points, and 6500 Hz in F2 and 15000 Hz in F1. The time between repetitions was 1.5 seconds for these experiments. Data were processed using the Varian VNMR software, version 6.1 C. The final data size, after linear-prediction in (t₁) and zero-filling in both dimensions, was 1024(F1)×2048(F2) complex points for all experiments.

### EXAMPLES

1. Oxygenase Protein and Nucleic Acid Sequences

As described above, the invention provides oxygenases and oxygenase-specific nucleic acid sequences. With the provision herein of these oxygenase sequences, the polymerase chain reaction (PCR) may be utilized as a preferred method for identifying and producing nucleic acid sequences encoding the oxygenases. For example, PCR amplification of the oxygenase sequences may be accomplished either by direct PCR from a plant cDNA library or by Reverse-Transcription PCR (RT-PCR) using RNA extracted from plant cells as a template. Oxygenase sequences may be amplified from plant genomic libraries, or plant genomic DNA. Methods and conditions for both direct PCR and RT-PCR are known in the art and are described in Innis et al., PCR Protocols: A Guide to Methods and Applications, Academic Press: San Diego, 1990.

The selection of PCR primers can be made according to the portions of the cDNA (or gene) that are to be amplified. Primers can be chosen to amplify small segments of the cDNA, the open reading frame, the entire cDNA molecule, or the entire gene sequence. Variations in amplification conditions may be required to accommodate primers of differing lengths; such considerations are well known in the art and are discussed in Innis et al., PCR Protocols. A Guide to Methods and Applications, Academic Press: San Diego, 1990; Sambrook et al. (eds.), Molecular Cloning: A Laboratory Manual, 2nd ed., vols. 1-3, Cold Spring Harbor Laboratory Press, Cold Spring Harbor, N.Y., 1989; and Ausubel et al. (eds.) Current Protocols in Molecular Biology, Greene Publishing and Wiley-Interscience, New York (with periodic updates), 1987. By way of example, the cDNA molecules corresponding to additional oxygenases may be amplified using primers directed toward regions of homology between the 5′ and 3′ ends of the full-length clone such as the one shown in SEQ ID NO: 43 sequences. Example primers for such a reaction are:

primer 1: 5′-CCI CCI GGI AAI ITI-3′ (SEQ ID NO: 81)

primer 2: 5′-ICC I(G/C)C ICC (G/A)AA IGG-3′ (SEQ ID NO: 82)

These primers are illustrative only; it will be appreciated by one skilled in the art that many different primers may be derived from the provided nucleic acid sequences. Re-sequencing of PCR products obtained by these amplification procedures is recommended to facilitate confirmation of the amplified sequence and to provide information on natural variation between oxygenase sequences. Oligonucleotides derived from the oxygenase sequence may be used in such sequencing methods.

Oligonucleotides that are derived from the oxygenase sequences are encompassed within the scope of the present invention. Preferably, such oligonucleotide primers comprise a sequence of at least 10-20 consecutive nucleotides of the oxygenase sequences. To enhance amplification specificity, oligonucleotide primers comprising at least 15, 20, 25, 30, 35, 40, 45 or 50 consecutive nucleotides of these sequences also may be used.

A. Oxygenases in Other Plant Species

Orthologs of the oxygenase genes are present in a number of other members of the Taxus genus. With the provision herein of the oxygenase nucleic acid sequences, the cloning by standard methods of cDNAs and genes that encode oxygenase orthologs in these other species is now enabled. As described above, orthologs of the disclosed oxygenase genes have oxygenase biological activity and are typically characterized by possession of at least 50% sequence identity counted over the full-length alignment with the amino acid sequence of the disclosed oxygenase sequences using the NCBI Blast 2.0 (gapped blastp set to default parameters). Proteins with even greater sequence identity to the reference sequences will show increasing percentage identities when assessed by this method, such as at least 60%, at least 65%, at least 70%, at least 75%, at least 80%, at least 90%, or at least 95% sequence identity.

Both conventional hybridization and PCR amplification procedures may be utilized to clone sequences encoding oxygenase orthologs. Common to both of these techniques is the hybridization of probes or primers that are derived from the oxygenase nucleic acid sequences. Furthermore, the hybridization may occur in the context of Northern blots, Southern blots, or PCR.

Direct PCR amplification may be performed on cDNA or genomic libraries prepared from the plant species in question, or RT-PCR may be performed using mRNA extracted from the plant cells using standard methods. PCR primers will comprise at least ten consecutive nucleotides of the oxygenase sequences. One of skill in the art will appreciate that sequence differences between the oxygenase nucleic acid sequence and the target nucleic acid to be amplified may result in lower amplification efficiencies. To compensate for this, longer PCR primers or lower annealing temperatures may be used during the amplification cycle. Whenever lower annealing temperatures are used, sequential rounds of amplification using nested primer pairs may be necessary to enhance specificity.

For conventional hybridization techniques the hybridization probe is preferably conjugated with a detectable label such as a radioactive label, and the probe is preferably at least ten nucleotides in length. As is well known in the art, increasing the length of hybridization probes tends to give enhanced specificity. The labeled probe derived from the oxygenase nucleic acid sequence may be hybridized to a plant cDNA or genomic library and the hybridization signal detected using methods known in the art. The hybridizing colony or plaque (depending on the type of library used) is purified and the cloned sequence contained in that colony or plaque isolated and characterized.

Orthologs of the oxygenases alternatively may be obtained by immunoscreening of an expression library. With the provision herein of the disclosed oxygenase nucleic acid sequences, the enzymes may be expressed and purified in a heterologous expression system (e.g., E. coli) and used to raise antibodies (monoclonal or polyclonal) specific for oxygenases. Antibodies also may be raised against synthetic peptides derived from the oxygenase amino acid sequence presented herein. Methods of raising antibodies are well known in the art and are described generally in Harlow and Lane, Antibodies, A Laboratory Manual, Cold Springs Harbor, 1988. Such antibodies can be used to screen an expression cDNA library produced from a plant. This screening will identify the oxygenase ortholog. The selected cDNAs can be confirmed by sequencing and enzyme activity assays.

B. Taxol® Oxygenase Variants

With the provision of the oxygenase amino acid sequences (SEQ ID NOS: 56-68) and the corresponding cDNA (SEQ ID NOS: 43-55 and 81-86), variants of these sequences now can be created.

Variant oxygenases include proteins that differ in amino acid sequence from the oxygenase sequences disclosed, but that retain oxygenase biological activity. Such proteins may be produced by manipulating the nucleotide sequence encoding the oxygenase using standard procedures such as site-directed mutagenesis or the polymerase chain reaction. The simplest modifications involve the substitution of one or more amino acids for amino acids having similar biochemical properties. These so-called “conservative substitutions” are likely to have minimal impact on the activity of the resultant protein. Table 4 shows amino acids that may be substituted for an original amino acid in a protein and that are regarded as conservative substitutions.

TABLE 4

| Original | Conservative |
|---|---|
| Residue | Substitutions |
| ala | Ser |
| arg | Lys |
| asn | Gln; his |
| asp | Glu |
| cys | Ser |
| gln | Asn |
| glu | Asp |
| gly | Pro |
| his | Asn; gln |
| ile | Leu; val |
| leu | ile; val |
| lys | Arg; gln; glu |
| met | Leu; ile |
| phe | Met; leu; tyr |
| ser | Thr |
| thr | Ser |
| trp | Tyr |
| tyr | Trp; phe |
| val | ile; leu |

More substantial changes in enzymatic function or other features may be obtained by selecting substitutions that are less conservative than those in Table 4, i.e., by selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions that in general are expected to produce the greatest changes in protein properties will be those in which: (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine. The effects of these amino acid substitutions or deletions or additions may be assessed for oxygenase derivatives by analyzing the ability of the derivative proteins to catalyse the conversion of one Taxol® precursor to another Taxol® precursor.

Variant oxygenase cDNA or genes may be produced by standard DNA-mutagenesis techniques, for example, M13 primer mutagenesis. Details of these techniques are provided in Sambrook et al. (eds.), Molecular Cloning: A Laboratory Manual, 2nd ed., vols. 1-3, Cold Spring Harbor Laboratory Press, Cold Spring Harbor, N.Y., 1989, Ch. 15. By the use of such techniques, variants may be created that differ in minor ways from the oxygenase cDNA or gene sequences, yet that still encode a protein having oxygenase biological activity. DNA molecules and nucleotide sequences that are derivatives of those specifically disclosed herein and that differ from those disclosed by the deletion, addition, or substitution of nucleotides while still encoding a protein having oxygenase biological activity are comprehended by this invention. In their simplest form, such variants may differ from the disclosed sequences by alteration of the coding region to fit the codon usage bias of the particular organism into which the molecule is to be introduced.

Alternatively, the coding region may be altered by taking advantage of the degeneracy of the genetic code to alter the coding sequence in such a way that, while the nucleotide sequence is substantially altered, it nevertheless encodes a protein having an amino acid sequence identical or substantially similar to the disclosed oxygenase amino acid sequences. For example, the nineteenth amino acid residue of the oxygenase (Clone F12, SEQ ID NO: 43) is alanine. This is encoded in the open reading frame (ORF) by the nucleotide codon triplet GCT. Because of the degeneracy of the genetic code, three other nucleotide codon triplets—GCA, GCC, and GCG—also code for alanine. Thus, the nucleotide sequence of the ORF can be changed at this position to any of these three codons without affecting the amino acid composition of the encoded protein or the characteristics of the protein. Based upon the degeneracy of the genetic code, variant DNA molecules may be derived from the cDNA and gene sequences disclosed herein using standard DNA-mutagenesis techniques as described above, or by synthesis of DNA sequences. Thus, this invention also encompasses nucleic acid sequences that encode the oxygenase protein but that vary from the disclosed nucleic acid sequences by virtue of the degeneracy of the genetic code.

Variants of the oxygenase also may be defined in terms of their sequence identity with the oxygenase amino acid (SEQ ID NOS: 56-68 and 87-92) and nucleic acid sequences (SEQ ID NOS: 43-55 and 81-86). As described above, oxygenases have oxygenase biological activity and share at least 60% sequence identity with the disclosed oxygenase sequences. Nucleic acid sequences that encode such proteins may be determined readily simply by applying the genetic code to the amino acid sequence of the oxygenase, and such nucleic acid molecules may be produced readily by assembling oligonucleotides corresponding to portions of the sequence.

As previously mentioned, another method of identifying variants of the oxygenases is nucleic acid hybridization. Nucleic acid molecules derived from the oxygenase cDNA and gene sequences include molecules that hybridize under various conditions to the disclosed Taxol® oxygenase nucleic acid molecules, or fragments thereof.

Nucleic acid duplex or hybrid stability is expressed as the melting temperature at which a probe dissociates from a target DNA. This melting temperature is used to define the required stringency conditions. If sequences are to be identified that are related and substantially identical to the probe, rather than identical, then it is useful first to establish the lowest temperature at which only homologous hybridization occurs with a particular concentration of salt (e.g., SSC or SSPE). Then, assuming that 1% mismatching results in a 1° C. decrease in the Tₘ, the temperature of the final wash in the hybridization reaction is reduced accordingly (for example, if sequences having >95% identity with the probe are sought, the final wash temperature is decreased by 5° C.). In practice, the change in Tₘ can be between 0.5° C. and 1.5° C. per 1% mismatch.

Generally, hybridization conditions are classified into categories, for example very high stringency, high stringency, and low stringency. The conditions for probes that are about 600 base pairs or more in length are provided below in three corresponding categories.

Very High Stringency (sequences greater than
90% sequence identity)

| | | | | | |
|---|---|---|---|---|---|
| Hybridization in | 5x | SSC | at | 65° C. | 16 hours |
| Wash twice in | 2x | SSC | at | room temp. | 15 minutes each |
| Wash twice in | 2x | SSC | at | 55° C. | 20 minutes each |

High Stringency (detects sequences that share approximately
80% sequence identity)

| | | | | | |
|---|---|---|---|---|---|
| Hybridization in | 5x | SSC | at | 42° C. | 16 hours |
| Wash twice in | 2x | SSC | at | room temp. | 20 minutes each |
| Wash once in | 2x | SSC | at | 42° C. | 30 minutes each |

Low Stringency (detects sequences that share 70% sequence
identity or greater)

| | | | | | |
|---|---|---|---|---|---|
| Hybridization in | 6x | SSC | at | room temp. | 16 hours |
| Wash twice in | 2x | SSC | at | room temp. | 20 minutes each |

The sequences encoding the oxygenases identified through hybridization may be incorporated into transformation vectors and introduced into host cells to produce the respective oxygenase.

2. Introduction of Oxygenases into Plants

After a cDNA (or gene) encoding a protein involved in the determination of a particular plant characteristic has been isolated, standard techniques may be used to express the cDNA in transgenic plants in order to modify the particular plant characteristic. The basic approach is to clone the cDNA into a transformation vector, such that the cDNA is operably linked to control sequences (e.g., a promoter) directing expression of the cDNA in plant cells. The transformation vector is introduced into plant cells by any of various techniques (e.g., electroporation), and progeny plants containing the introduced cDNA are selected. Preferably all or part of the transformation vector stably integrates into the genome of the plant cell. The portion of the transformation vector that integrates into the plant cell and that contains the introduced cDNA and associated sequences for controlling expression (the introduced “transgene”) may be referred to as the recombinant expression cassette.

Selection of progeny plants containing the introduced transgene may be made based upon the detection of an altered phenotype. Such a phenotype may result directly from the cDNA cloned into the transformation vector or may be manifest as enhanced resistance to a chemical agent (such as an antibiotic) as a result of the inclusion of a dominant selectable marker gene incorporated into the transformation vector.

Successful examples of the modification of plant characteristics by transformation with cloned cDNA sequences are replete in the technical and scientific literature. Selected examples serving to illustrate the knowledge in this field of technology include:

U.S. Pat. No. 5,571,706 (“Plant Virus Resistance Gene and Methods”)

U.S. Pat. No. 5,677,175 (“Plant Pathogen Induced Proteins”)

U.S. Pat. No. 5,510,471 (“Chimeric Gene for the Transformation of Plants”)

U.S. Pat. No. 5,750,386 (“Pathogen-Resistant Transgenic Plants”)

U.S. Pat. No. 5,597,945 (“Plants Genetically Enhanced for Disease Resistance”)

U.S. Pat. No. 5,589,615 (“Process for the Production of Transgenic Plants with Increased Nutritional Value Via the Expression of Modified 2S Storage Albumins”)

U.S. Pat. No. 5,750,871 (“Transformation and Foreign Gene Expression in Brassica Species”)

U.S. Pat. No. 5,268,526 (“Overexpression of Phytochrome in Transgenic Plants”)

U.S. Pat. No. 5,262,316 (“Genetically Transformed Pepper Plants and Methods for their Production”)

U.S. Pat. No. 5,569,831 (“Transgenic Tomato Plants with Altered Polygalacturonase Isoforms”)

These examples include descriptions of transformation-vector selection, transformation techniques, and the construction of constructs designed to over-express the introduced cDNA. In light of the foregoing and the provision herein of the oxygenase amino acid sequences and nucleic acid sequences, it is thus apparent that one of skill in the art will be able to introduce the cDNAs, or homologous or derivative forms of these molecules, into plants in order to produce plants having enhanced oxygenase activity. Furthermore, the expression of one or more oxygenases in plants may give rise to plants having increased production of Taxol® and related compounds.

A. Vector Construction, Choice of Promoters

A number of recombinant vectors suitable for stable transfection of plant cells or for the establishment of transgenic plants have been described, including those described in Weissbach and Weissbach, Methods for Plant Molecular Biology, Academic Press, 1989; and Gelvin et al., Plant and Molecular Biology Manual, Kluwer Academic Publishers, 1990. Typically, plant-transformation vectors include one or more cloned plant genes (or cDNAs) under the transcriptional control of 5′- and 3′-regulatory sequences and a dominant selectable marker. Such plant-transformation vectors typically also contain a promoter regulatory region (e.g., a regulatory region controlling inducible or constitutive, environmentally or developmentally regulated, or cell- or tissue-specific expression), a transcription-initiation start site, a ribosome-binding site, an RNA processing signal, a transcription-termination site, and/or a polyadenylation signal.

Examples of constitutive plant promoters that may be useful for expressing the cDNA include: the cauliflower mosaic virus (CaMV) 35S promoter, which confers constitutive, high-level expression in most plant tissues (see, e.g., Odel et al., Nature 313:810, 1985; Dekeyser et al., Plant Cell 2:591, 1990; Terada and Shimamoto, Mol. Gen. Genet. 220:389, 1990; and Benfey and Chua, Science 250:959-966, 1990); the nopaline synthase promoter (An et al., Plant Physiol. 88:547, 1988); and the octopine synthase promoter (Fromm et al., Plant Cell 1:977, 1989). Agrobacterium-mediated transformation of Taxus species has been accomplished, and the resulting callus cultures have been shown to produce Taxol® (Han et al., Plant Science 95:187-196, 1994). Therefore, it is likely that incorporation of one or more of the described oxygenases under the influence of a strong promoter (like CaMV promoter) would increase production yields of Taxol® and related taxoids in such transformed cells.

A variety of plant gene promoters that are regulated in response to environmental, hormonal, chemical, and/or developmental signals also can be used for expression of the cDNA in plant cells, including promoters regulated by: (a) heat (Callis et al., Plant Physiol. 88:965, 1988; Ainley, et al., Plant Mol. Biol. 22:13-23, 1993; and Gilmartin et al., The Plant Cell 4:839-949, 1992); (b) light (e.g., the pea rbcS-3A promoter, Kuhlemeier et al., Plant Cell 1:471, 1989, and the maize rbcS promoter, Schaffner and Sheen, Plant Cell 3:997, 1991); (c) hormones, such as abscisic acid (Marcotte et al., Plant Cell 1:969, 1989); (d) wounding (e.g., wunI, Siebertz et al., Plant Cell 1:961, 1989); and (e) chemicals such as methyl jasmonate or salicylic acid (see also Gatz et al., Ann. Rev. Plant Physiol. Plant Mol. Biol. 48:9-108, 1997).

Alternatively, tissue-specific (root, leaf, flower, and seed, for example) promoters (Carpenter et al., The Plant Cell 4:557-571, 1992; Denis et al., Plant Physiol. 101:1295-1304, 1993; Opperman et al., Science 263:221-223, 1993; Stockhause et al., The Plant Cell 9:479-489, 1997; Roshal et al., Embo. J. 6:1155, 1987; Schernthaner et al., Embo J. 7:1249, 1988; and Bustos et al., Plant Cell 1:839, 1989) can be fused to the coding sequence to obtain a particular expression in respective organs.

Alternatively, the native oxygenase gene promoters can be utilized. With the provision herein of the oxygenase nucleic acid sequences, standard molecular biology techniques can be used to determine the corresponding promoter sequences, less than the entire promoter sequence can be used in order to obtain effective promoter activity. The determination of whether a particular region of this sequence confers effective promoter activity may be ascertained readily by operably linking the selected sequence region to an oxygenase cDNA (in conjunction with suitable 3′ regulatory region, such as the NOS 3′ regulatory region as discussed below) and determining whether the oxygenase is expressed.

Plant-transformation vectors also may include RNA processing signals, for example, introns, that may be positioned upstream or downstream of the ORF sequence in the transgene. In addition, the expression vectors also may include additional regulatory sequences from the 3′-untranslated region of plant genes, e.g., a 3′-terminator region, to increase mRNA stability of the mRNA, such as the PI-II terminator region of potato or the octopine or nopaline synthase (NOS) 3′-terminator regions. The native oxygenase gene 3′-regulatory sequence also may be employed.

Finally, as noted above, plant-transformation vectors also may include dominant selectable marker genes to allow for the ready selection of transformants. Such genes include those encoding antibiotic-resistance genes (e.g., resistance to hygromycin, kanamycin, bleomycin, G418, streptomycin, or spectinomycin) and herbicide-resistance genes (e.g., phosphinothricin acetyloxygenase).

B. Arrangement of Taxol® oxygenase Sequence in a Vector

The particular arrangement of the oxygenase sequence in the transformation vector is selected according to the type of expression of the sequence that is desired.

In most instances, enhanced oxygenase activity is desired, and the oxygenase ORF is operably linked to a constitutive high-level promoter such as the CaMV 35S promoter. As noted above, enhanced oxygenase activity also may be achieved by introducing into a plant a transformation vector containing a variant form of the oxygenase cDNA or gene, for example a form that varies from the exact nucleotide sequence of the oxygenase orf, but that encodes a protein retaining an oxygenase biological activity.

C. Transformation and Regeneration Techniques

Transformation and regeneration of both monocotyledonous and dicotyledonous plant cells are now routine, and the appropriate transformation technique can be determined by the practitioner. The choice of method varies with the type of plant to be transformed; those skilled in the art will recognize the suitability of particular methods for given plant types. Suitable methods may include, but are not limited to: electroporation of plant protoplasts, liposome-mediated transformation, polyethylene glycol (PEG)-mediated transformation, transformation using viruses, micro-injection of plant cells, micro-projectile bombardment of plant cells, vacuum infiltration, and Agrobacterium tumefaciens (AT)-mediated transformation. Typical procedures for transforming and regenerating plants are described in the patent documents listed at the beginning of this section.

D. Selection of Transformed Plants

Following transformation and regeneration of plants with the transformation vector, transformed plants can be selected using a dominant selectable marker incorporated into the transformation vector. Typically, such a marker confers antibiotic resistance on the seedlings of transformed plants, and selection of transformants can be accomplished by exposing the seedlings to appropriate concentrations of the antibiotic.

After transformed plants are selected and grown to maturity, they can be assayed using the methods described herein to assess production levels of Taxol® and related compounds.

3. Production of Recombinant Taxol® Oxygenase in Heterologous Expression Systems

Various yeast strains and yeast-derived vectors are used commonly for the expression of heterologous proteins. For instance, Pichia pastoris expression systems, obtained from Invitrogen (Carlsbad, Calif.), may be used to practice the present invention. Such systems include suitable Pichia pastoris strains, vectors, reagents, transformants, sequencing primers, and media. Available strains include KM71H (a prototrophic strain), SMD1168H (a prototrophic strain), and SMD1168 (a pep4 mutant strain) (Invitrogen Product Catalogue, 1998, Invitrogen, Carlsbad Calif.).

Non-yeast eukaryotic vectors may be used with equal facility for expression of proteins encoded by modified nucleotides according to the invention. Mammalian vector/host cell systems containing genetic and cellular control elements capable of carrying out transcription, translation, and post-translational modification are well known in the art. Examples of such systems are the well-known baculovirus system, the ecdysone-inducible expression system that uses regulatory elements from Drosophila melanogaster to allow control of gene expression, and the sindbis viral-expression system that allows high-level expression in a variety of mammalian cell lines, all of which are available from Invitrogen, Carlsbad, Calif.

The cloned expression vector encoding one or more oxygenases may be transformed into any of various cell types for expression of the cloned nucleotide. Many different types of cells may be used to express modified nucleic acid molecules. Examples include cells of yeasts, fungi, insects, mammals, and plants, including transformed and non-transformed cells. For instance, common mammalian cells that could be used include HeLa cells, SW-527 cells (ATCC deposit #7940), WISH cells (ATCC deposit #CCL-25), Daudi cells (ATCC deposit #CCL-213), Mandin-Darby bovine kidney cells (ATCC deposit #CCL-22) and Chinese hamster ovary (CHO) cells (ATCC deposit #CRL-2092). Common yeast cells include Pichia pastoris (ATCC deposit #201178) and Saccharomyces cerevisiae (ATCC deposit #46024). Insect cells include cells from Drosophila melanogaster (ATCC deposit #CRL-1019 1), the cotton bollworm (ATCC deposit #CRL-9281), and Trichoplusia ni egg cell homoflagellates. Fish cells that may be used include those from rainbow trout (ATCC deposit #CLL-55), salmon (ATCC deposit #CRL-1681), and zebrafish (ATCC deposit #CRL-2147). Amphibian cells that may be used include those of the bullfrog, Rana catesbelana (ATCC deposit #CLL-41). Reptile cells that may be used include those from Russell's viper (ATCC deposit #CCL-140). Plant cells that could be used include Chlamydomonas cells (ATCC deposit #30485), Arabidopsis cells (ATCC deposit #54069) and tomato plant cells (ATCC deposit #54003). Many of these cell types are commonly used and are available from the ATCC as well as from commercial suppliers such as Pharmacia (Uppsala, Sweden), and Invitrogen.

Expressed protein may be accumulated within a cell or may be secreted from the cell. Such expressed protein may then be collected and purified. This protein may be characterized for activity and stability and may be used to practice any of the various methods according to the invention.

4. Creation of Oxygenase-Specific Binding Agents

Antibodies to the oxygenase enzymes, and fragments thereof, of the present invention may be useful for purification of the enzymes. The provision of the oxygenase sequences allows for the production of specific antibody-based binding agents to these enzymes.

Monoclonal or polyclonal antibodies may be produced to an oxygenase, portions of the oxygenase, or variants thereof. Optimally, antibodies raised against epitopes on these antigens will detect the enzyme specifically. That is, antibodies raised against an oxygenase would recognize and bind the oxygenase, and would not substantially recognize or bind to other proteins. The determination that an antibody specifically binds to an antigen is made by any one of a number of standard immunoassay methods, e.g., Western blotting, Sambrook et al. (eds.), Molecular Cloning: A Laboratory Manual, 2nd ed., vols. 1-3, Cold Spring Harbor Laboratory Press, Cold Spring Harbor, N.Y., 1989.

To determine that a given antibody preparation (such as a preparation produced in a mouse against SEQ ID NO: 56) specifically detects the oxygenase by Western blotting, total cellular protein is extracted from cells and electrophoresed on an SDS-polyacrylamide gel. The proteins are transferred to a membrane (e.g., nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase; application of 5-bromo-4-chloro-3-indolyl phosphate/nitro blue tetrazolium results in the production of a densely blue-colored compound by immuno-localized alkaline phosphatase.

Antibodies that specifically detect an oxygenase will be shown, by this technique, to bind substantially only the oxygenase band (having a position on the gel determined by the molecular weight of the oxygenase). Non-specific binding of the antibody to other proteins may occur and may be detectable as a weaker signal on the Western blot (which can be quantified by automated radiography). The non-specific nature of this binding will be recognized by one skilled in the art by the weak signal obtained on the Western blot relative to the strong primary signal arising from the specific anti-oxygenase binding.

Antibodies that specifically bind to an oxygenase according to the invention belong to a class of molecules that are referred to herein as “specific binding agents.” Specific binding agents capable of specifically binding to the oxygenase of the present invention may include polyclonal antibodies, monoclonal antibodies and fragments of monoclonal antibodies such as Fab, F(ab′)₂ and Fv fragments, as well as any other agent capable of specifically binding to one or more epitopes on the proteins.

Substantially pure oxygenase suitable for use as an immunogen can be isolated from transfected cells, transformed cells, or from wild-type cells. Concentration of protein in the final preparation is adjusted, for example, by concentration on an Amicon filter device, to the level of a few micrograms per milliliter. Alternatively, peptide fragments of an oxygenase may be utilized as immunogens. Such fragments may be synthesized chemically using standard methods, or may be obtained by cleavage of the whole oxygenase enzyme followed by purification of the desired peptide fragments. Peptides as short as three or four amino acids in length are immunogenic when presented to an immune system in the context of a Major Histocompatibility Complex (MHC) molecule, such as MHC class I or MHC class II. Accordingly, peptides comprising at least 3 and preferably at least 4, 5, 6 or more consecutive amino acids of the disclosed oxygenase amino acid sequences may be employed as immunogens for producing antibodies.

Because naturally occurring epitopes on proteins frequently comprise amino acid residues that are not adjacently arranged in the peptide when the peptide sequence is viewed as a linear molecule, it may be advantageous to utilize longer peptide fragments from the oxygenase amino acid sequences for producing antibodies. Thus, for example, peptides that comprise at least 10, 15, 20, 25, or 30 consecutive amino acid residues of the amino acid sequence may be employed. Monoclonal or polyclonal antibodies to the intact oxygenase, or peptide fragments thereof may be prepared as described below.

A. Monoclonal Antibody Production by Hybridoma Fusion

Monoclonal antibody to any of various epitopes of the oxygenase enzymes that are identified and isolated as described herein can be prepared from murine hybridomas according to the classic method of Kohler & Milstein, Nature 256:495, 1975, or a derivative method thereof. Briefly, a mouse is repetitively inoculated with a few micrograms of the selected protein over a period of a few weeks. The mouse is sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA (enzyme-linked immunosorbent assay, as originally described by Engvall, Enzymol. 70:419, 1980) or a derivative method thereof. Selected positive clones can be expanded and their monoclonal antibody product harvested for use. Detailed procedures for monoclonal antibody production are described in Harlow & Lane, Antibodies, A Laboratory Manual, Cold Spring Harbor Laboratory, New York, 1988.

B. Polyclonal Antibody Production by Immunization

Polyclonal antiserum containing antibodies to heterogenous epitopes of a single protein can be prepared by immunizing suitable animals with the expressed protein, which can be unmodified or modified, to enhance immunogenicity. Effective polyclonal antibody production is affected by many factors related both to the antigen and the host species. For example, small molecules tend to be less immunogenic than other molecules and may require the use of carriers and an adjuvant. Also, host animals vary in response to site of inoculations and dose, with both inadequate or excessive doses of antigen resulting in low-titer antisera. Small doses (ng level) of antigen administered at multiple intradermal sites appear to be most reliable. An effective immunization protocol for rabbits can be found in Vaitukaitis et al., J. Clin. Endocrinol. Metab. 33:988-991, 1971.

Booster injections can be given at regular intervals, and antiserum harvested when the antibody titer thereof, as determined semi-quantitatively, for example, by double immunodiffusion in agar against known concentrations of the antigen, begins to fall. See, e.g., Ouchterlony et al., in Wier (ed.), Handbook of Experimental Immunology, Chapter 19, Blackwell, 1973. A plateau concentration of antibody is usually in the range of 0.1 to 0.2 mg/mL of serum (about 12 μM). Affinity of the antisera for the antigen is determined by preparing competitive binding curves using conventional methods.

C. Antibodies Raised by Injection of cDNA

Antibodies may be raised against an oxygenase of the present invention by subcutaneous injection of a DNA vector that expresses the enzymes in laboratory animals, such as mice. Delivery of the recombinant vector into the animals may be achieved using a hand-held form of the “Biolistic” system (Sanford et al., Particulate Sci. Technol. 5:27-37, 1987, as described by Tang et al., Nature (London) 356:153-154, 1992). Expression vectors suitable for this purpose may include those that express the cDNA of the enzyme under the transcriptional control of either the human β-actin promoter or the cytomegalovirus (CMV) promoter. Methods of administering naked DNA to animals in a manner resulting in expression of the DNA in the body of the animal are well known and are described, for example, in U.S. Pat. Nos. 5,620,896 (“DNA Vaccines Against Rotavirus Infections”); 5,643,578 (“Immunization by Inoculation of DNA Transcription Unit”); and 5,593,972 (“Genetic Immunization”), and references cited therein.

D. Antibody Fragments

Antibody fragments may be used in place of whole antibodies and may be readily expressed in prokaryotic host cells. Methods of making and using immunologically effective portions of monoclonal antibodies, also referred to as “antibody fragments,” are well known and include those described in Better & Horowitz, Methods Enzymol. 178:476-496, 1989; Glockshuber et al. Biochemistry 29:1362-1367, 1990; and U.S. Pat. Nos. 5,648,237 (“Expression of Functional Antibody Fragments”); 4,946,778 (“Single Polypeptide Chain Binding Molecules”); and 5,455,030 (“Immunotherapy Using Single Chain Polypeptide Binding Molecules”), and references cited therein.

5. Taxol® Production in vivo

The creation of recombinant vectors and transgenic organisms expressing the vectors are important for controlling the production of oxygenases. These vectors can be used to decrease oxygenase production, or to increase oxygenase production. A decrease in oxygenase production likely will result from the inclusion of an antisense sequence or a catalytic nucleic acid sequence that targets the oxygenase encoding nucleic acid sequence. Conversely, increased production of oxygenase can be achieved by including at least one additional oxygenase encoding sequence in the vector. These vectors can be introduced into a host cell, thereby altering oxygenase production. In the case of increased production, the resulting oxygenase may be used in in vitro systems, as well as in vivo for increased production of Taxol®, other taxoids, intermediates of the Taxol® biosynthetic pathway, and other products.

Increased production of Taxol® and related taxoids in vivo can be accomplished by transforming a host cell, such as one derived from the Taxus genus, with a vector containing one or more nucleic acid sequences encoding one or more oxygenases. Furthermore, the heterologous or homologous oxygenase sequences can be placed under the control of a constitutive promoter, or an inducible promoter. This will lead to the increased production of oxygenase, thus eliminating any rate-limiting effect on Taxol® production caused by the expression and/or activity level of the oxygenase.

6. Taxol® Production in vitro

Currently, Taxol is produced by a semisynthetic method described in Hezari and Croteau, Planta Medica 63:291-295, 1997. This method involves extracting 10-deacetyl-baccatin III, or baccatin III, intermediates in the Taxol® biosynthetic pathway, and then finishing the production of Taxol® using in vitro techniques. As more enzymes are identified in the Taxol® biosynthetic pathway, it may become possible to completely synthesize Taxol® in vitro, or at least to increase the number of steps that can be performed in vitro. Hence, the oxygenases of the present invention may be used to facilitate the production of Taxol® and related taxoids in synthetic or semi-synthetic methods. Accordingly, the present invention enables the production of not only transgenic organisms that produce increased levels of Taxol®, but also transgenic organisms that produce increased levels of important intermediates, such as 10-deacetyl-baccatin III and baccatin III.

7. Alternative Substrates for use in Assessing Taxoid Oxygenases Activity

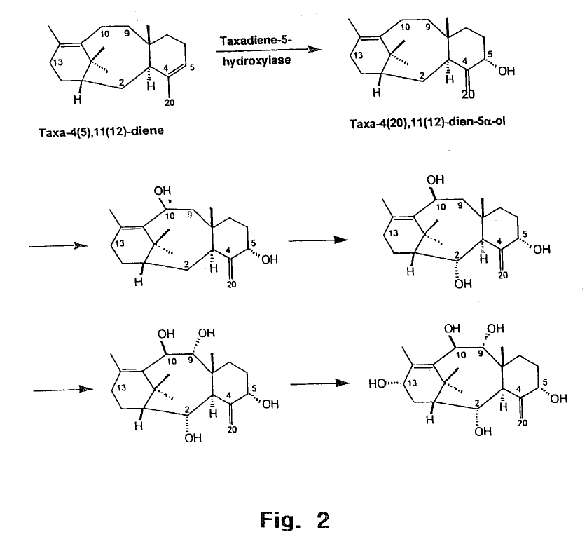

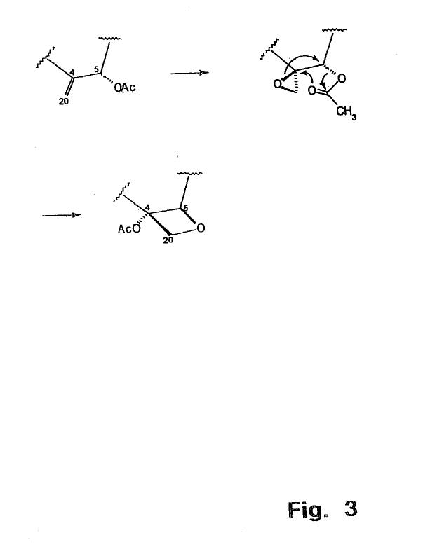

The order of oxygenation reactions on the taxane (taxadiene) nucleus en route to Taxol® is not precisely known. However, based on comparison of the structures of the several hundred naturally-occurring taxanes (Kingston et al., The Taxane Diterpenoids, in Herz et al. (eds.), Progress in the Chemistry of Organic Natural Products, Springer-Verlag, N.Y., vol. 61, p. 206, 1993; and Baloglu et al., J. Nat. Prod. 62:1448-1472, 1999), it can be deduced from relative abundances of taxoids with oxygen substitution at each position (Floss et al., Biosynthesis of Taxol, in Suffness (ed.), Taxol: Science and Applications, CRC Press, Boca Raton, Fla., pp. 191-208, 1995) that oxygens at C5 (carbon numbers shown in FIG. 2) and C10 are introduced first, followed by oxygenation at C2 and C9 (could be either order), then at C13. Oxygenations at C7 and C1 of the taxane nucleus are considered to be very late introductions, possibly occurring after oxetane ring formation; however, epoxidation (at C4/C20) and oxetane formation seemingly must precede oxidation of the C9 hydroxyl to a carbonyl (Floss et al., Biosynthesis of Taxol, in Suffness (ed.), Taxol: Science and Applications, CRC Press, Boca Raton, Fla., pp. 191-208, 1995). Evidence from cell-free enzyme studies with Taxus microsomes (Hezari et al., Planta Medica 63:291-295, 1997) and in vivo feeding studies with Taxus cells (Eisenreich et al., J. Am. Chem. Soc. 120:9694-9695, 1998) have indicated that the oxygenation reactions of the taxane core are accomplished by cytochrome P450 oxygenases. Thus, for example, the cytochrome P450-mediated hydroxylation (with double-bond migration) of taxadiene to taxadien-5α-ol has been demonstrated with Taxus microsomes (Hefner et al., Chem. Biol. 3:479-489, 1996). Most recently, the taxadien-5α-ol (and acetate ester thereof) have been shown to undergo microsomal P450-catalyzed oxygenation to the level of a pentaol (i.e., taxadien-2α,5α,9α,10β,13α-pentaol) (Hezari et al., Planta Medica 63:291-295, 1997).

Because downstream steps are not yet defined, the above-referenced research summarized in Table 2 involved the pursuit of reactions (the timing and regiochemistry (position) of subsequent taxoid hydroxylations) through the use of surrogate substrates. Thus, labeled (+)-taxusin (the tetraacetate of taxadien-5,9,10,13-tetraol) was utilized to evaluate hydroxylations at C1, C2 and C7, and the epoxidation at C4/C20 en route to formation of the oxetane D-ring of Taxol®.

Microsome preparations from Taxus cuspidata cells, optimized for cytochrome P450-mediated reactions, convert taxusin to the level of an epoxy triol (i.e., hydroxylation at C1, C2, and C7 and epoxidation of the C4/C20 double bond of the tetraacetate of taxadien-5,9,10,13-tetraol). Therefore, microsomal P450 reactions tentatively have been demonstrated for all of the relevant positions on the taxane core structure en route to Taxol® (C1, C2, C5, C7, C9, C10 and C13, and the C4/C20 epoxidation), although the exact order for the various positions has not been established firmly.

The screening of the functionally expressed (by CO-difference spectra) clones in yeast (using taxadienol and taxadienyl acetate as test substrates) demonstrated that clone F14 encodes the cytochrome P450 taxane-10β-hydroxylase. Similar screening of functionally expressed clones using baculovirus-Spodoptera (especially for clones that do not express well in yeast) also revealed clone F16 as encoding the cytochrome P450 taxane-13α-hydroxylase.

The remaining regiospecific (positionally specific) oxygenases that functionalize the taxane core en route to Taxol® can be obtained by identifying additional full-length clones by library screening with the appropriate hybridization probes or by RACE methods as necessary. Each clone can be functionally expressed (i.e., exhibiting a CO-difference spectrum which indicates proper folding and incorporation of heme) in yeast or Spodoptera, as necessary. Each expressed cytochrome P450 clone can be tested for catalytic capability by in vivo (in situ) and in vitro (isolated microsomes) assays with the various taxoid substrates as described below, using GC-MS and NMR methods to identify products and thereby establish the regiochemistry of hydroxylation of the taxane core. Suitable substrates for use in additional assays are provided in Table 5, below.

TABLE 5

| Substrate | Use |
|---|---|
| Taxa-4(20),11(12)-dien (taxadiene) | A radiolabeled synthetic substrate |
| | employed to search for 5α-hydroxylase. |
| Taxa-4(20),11(12)-dien-5α-ol and the | Radiolabeled synthetic substrates employed |
| corresponding 5α-acetate (taxadienol and | to search for early hydroxylation steps and |
| taxadienyl acetate) | to assist in sequencing the various |
| | regiospecific hydroxylations of the Taxol ® |
| | pathway. These substrates were employed |
| | to confirm the taxane 10β-hydroxylase |
| | (clone F14) and the taxane 13α- |
| | -hydroxylase (clone F16), and to indicate |
| | the early hydroxylation order as C5, C10 |
| | then C9. Preliminary evidence using these |
| | substrates suggests that clones F7, F9, F12, |
| | and F51 encode the C1, C2, C7, and C13 |
| | hydroxylases, respectivley, but the |
| | corresponding products (four different |
| | diols (and diol monoacetates)) have not |
| | been identified and the sequence of |
| | oxygenation following 10β-hydroxylation |
| | is not yet known. |
| Taxa-4(20),11(12)-dien-2α,5α-diol (and | Synthetic substrates used to search for the |
| diacetate ester) | C1, C7, and C13 hydroxylases and to assist |
| | in ordering the C2, C9, and C10 |
| | hydroxylation reactions of the pathway. |
| Taxa-4(20),11(12)-dien-5α,9α,10β,13α- | Radiolabeled, semisynthetic substrates |
| tetraol and corresponding tetraacetate | used to search for the C4/C20 epoxidase |
| (taxusin tetraol and taxusin, respectively) | and late-stage oxygenations, including C1 |
| | and C7 hydroxylases and the C2 |
| | hydroxylase. Also used to assist in ordering |
| | the late-stage oxygenation steps of the |
| | pathway. Although taxusin (and tetraol) do |
| | not reside on the Taxol ® pathway (Floss et |
| | al., Biosynthesis of Taxol, in Suffness (ed.), |
| | Taxol: Science and Applications, CRC |
| | Press, Boca Raton, FL, pp. 191-208, 1995), |
| | this surrogate substrate is metabolized to |
| | the level of a presumptive taxadien-4,20- |
| | epoxy-1,2,5,7,9,10,13-heptaol (and |
| | tetraacetate) by microsomal preparations, |
| | but structures of the reaction products have |
| | not yet been confirmed by NMR. |
| *Taxa-4(20),11(12)-dien-5α,13α-diol (and | Labeled biosynthetic substrates prepared |
| monoacetate and diacetate) | from taxadienol (and acetate) using the |
| | above-described clones (clone 16). Used in |
| | searching for and ordering downstream |
| | oxygenation reactions. |
| *Taxa-4(20),11(12)-dien-5α,10β-diol (and | Labeled biosynthetic substrates prepared |
| monoacetate and diacetate) | from taxadienol (and acetate) using the |
| | above-described clones (clone 14). Used in |
| | searching for and ordering downstream |
| | oxygenation reactions. |
| Taxa-4(20),11(12)-dien-5α,9α,10β-triol (an | Semisynthetic substrate prepared from |
| acetate esters) | taxusin, and used as in * above. |

Using these natural and surrogate substrates, along with the established expression methods and bioanalytical protocols, it is anticipated that all of the regiospecific cytochrome P450 taxoid oxygenases of the Taxol® pathway will be 5 acquired from the extant set of related cytochrome P450s.

8. Taxane 14β-Hydroxylase is a Cytochrome P450-Dependent Monooxygenase

Taxol® biosynthesis in Taxus (yew) species involves eight oxygenations at various positions of the core taxane ring system. The first reaction is hydroxylation at the C5 position of the committed precursor taxadiene to yield 5α-hydroxy taxadiene, and that this and subsequent regioselective hydroxylations are catalyzed by cytochrome P450 oxygenases. 5α-Hydroxy taxadiene is then either further oxygenated at C13 to yield 5α, 13α-dihydroxy taxadiene, or undergoes acetylation to 5α-acetoxy taxadiene before hydroxylation at C10 to form 5α-acetoxy-10β-hydroxy taxadiene.

To characterize other steps of the Taxol® biosynthetic pathway, some of the cytochrome P450 clones described herein were functionally expressed in yeast and screened by in vivo feeding of radiolabeled 5α-acetoxy-10β-hydroxy taxadiene and 5α,13α-dihydroxy taxadiene. One clone efficiently and specifically transformed the 5α-acetoxy-10β-ol, but not the 5α, 13α-diol, to a more polar product with the chromatographic properties of a taxane triol monoacetate, and the identity of this product was confirmed by spectroscopy as 5α-acetoxy-10β, 14β-dihydroxy taxadiene. Microsome preparation from the transformed yeast allowed characterization of this new hydroxylase, which was shown to resemble other cytochrome P450 taxoid hydroxylases having a pH optimum at 7.5 and a Kₘ value for the taxoid substrate of about 50 μM. Because Taxol® is unsubstituted at C14, this 14β-hydroxylase (Clone F72; SEQ ID NOS: 54 and 67) appears to be responsible for diversion of the pathway to 14-hydroxy taxoids that are prominent metabolites of Taxus cell cultures. Manipulation of this hydroxylase gene could permit redirection of the pathway to increase flux toward Taxol® and could allow the preparation of 13α, 14β-hydroxy taxoids as new therapeutic agents. Because Taxol® and related antineoplastic drugs, and the starting materials for their semisynthetic preparation, continue to be derived exclusively from biological sources (i.e., Taxus species), an understanding of the taxoid biosynthetic pathway, and its underlying molecular genetics, remains an important goal for the improvement of production yields of these drugs and their precursors. The oxygenation steps that sequentially functionalize the taxane core constitute a prominent feature of this complex biosynthetic pathway. There has been considerable recent interest in the semisynthesis of 13α, 14β-dihydroxy taxoids as Taxol®-like drugs with improved efficacy and bioavailability, potentially even as orally active forms. Although both 13α-hydroxy (the site of side-chain attachment) taxoids and 14β-hydroxy taxoids are prominent metabolites of various Taxus tissues, advanced taxoids bearing the vicinal 13α, 14β-diol function, as precursors for semisynthesis, are quite rare. This newly discovered taxoid 14β-hydroxylase, via modification of available 13α-hydroxy derivatives, could provide access to starting materials for the preparation of these “second generation” taxoid anticancer agents.

A. Materials and Methods—Enzymes, Substrates, and Reagents.

Enzymes and reagents were obtained from Invitrogen (Carlsbad, Calif.), New England Biolabs (Beverly, Mass.) and Stratagene (La Jolla, Calif.) as indicated, and were used according to the manufacturers' instructions. Chemicals were purchased from Merck (Darmstadt, Germany) and Sigma (St. Louis, Mo.). The preparations of [20-³H]taxa-4(5),11(12)-diene, [20-³H]5α-hydroxy taxa-4(20),11(12)-diene and [20-³H]5α-acetoxy taxa-4(20),11(12)-diene (all at 2 Ci/mol and prepared in racemic form) have been described. See, e.g., J. Hefner et al., Chem. and Biol. 3:479-489, 1996; Wheeler et al., Arch. Biochem. Biophys. 390:265-278, 2001; and Rubenstein et al., J. Label. Compd. Radiopharm. 43:481-491, 2000. [20-³H]5α-Acetoxy-10β-hydroxy taxa-4(20),11(12)-diene and [20-³H]5α, 13α-dihydroxy taxa-4(20),11(12)-diene (both at 2 Ci/mol) were obtained from [20-³H]5α-acetoxy taxa-4(20),11(12)-diene and [20-³H]5α-hydroxy taxa-4(20),11(12)-diene, respectively, via biotransformation using Saccharomyces cerevisiae WAT11 cells transformed with either the taxane 10β-hydroxylase or the taxane 13α-hydroxylase clone, respectively, and were isolated and purified as described below. See Pompon et al., Methods Enzymol. 272:51-64, 1996; Schoendorf et al., Proc. Natl. Acad. Sci. USA 98:1501-1506, 2001; and Jennewein et al., Proc. Natl. Acad. Sci. USA 98:13595-13600, 2001. The S. cerevisiae WAT11 cells, which express the ATR1 gene encoding an Arabidopsis thaliana-NADPH-cytochrome P450 reductase were obtained from Denis Pompon (Gif-sur-Yvette, France). See Urban, et al., J. Biol. Chem. 272:19176-19186, 1997; and Mizutani et al., Plant Physiol. 116:357-367, 1998.

B. Materials and Methods—Heterologous Expression of Cytochrome P450 Clones in Yeast.

For expression in S. cerevisiae WAT11 cells, the open reading frames (orf) of the previously isolated Taxus cuspidata cytochrome P450 clones were first amplified by standard PCR methods, but omitting the stop codons. The amplicons were gel-purified and transferred into the pYES 2.1 TOPO vector (Invitrogen), which was propagated in E. coli strain TOP10 F′ (Invitrogen). By omitting the stop codon, a C-terminal fusion between the cytochrome P450 orf and the vector-encoded V5 epitope and histidine (His₆) tag was created, thereby allowing sensitive detection of the expressed cytochrome P450 by immunoblot analysis of the isolated microsomal protein (see below). The 10β-hydroxylase and 13α-hydroxylase were employed as positive controls to confirm that the epitope-tagging did not influence hydroxylase activity, and β-galactosidase gene was identically installed in pYES 2.1 TOPO as a negative control for hydroxylase activity. The sequence-verified plasmids were then transformed into S. cerevisiae strain WAT11 using a lithium acetate method (Ito et al., J. Biol. Chem. 235: 2379-2385, 1983), and the yeast cells were cultured in YPLA medium until a cell density of 2—5×10⁸ cells per mL was reached in preparation for feeding studies or microsomal membrane isolation.

C. Materials and Methods—Assay of Recombinant Cytochrome P450 Oxygenases.

Following confirmation of expression by immunoblot analysis (see below), the activities of the recombinant cytochrome P450 enzymes were examined by in vivo feeding as before, thus eliminating the uncertainties associated with microsome isolation and assay. For this purpose, the transformed yeast cells were grown to stationary phase in 2 mL of SGIA medium at 30° C. under 250-rpm agitation. The cells were harvested by centrifugation, and the cell pellet was suspended in 3 mL of YPLA medium. Approximately 7 to 8 h after induction, the cells were harvested again by centrifugation and resuspended in 3 mL of fresh YPLA medium to which 30 μM of the labeled test substrate was added, followed by overnight incubation at 30° C. with agitation (250 rpm). The incubation mixture was then treated for 15 min in a sonication bath and extracted twice with 3 mL of hexane:ethyl acetate (4:1 v/v). The organic extract was then dried under N₂, the residue dissolved in 100 μL of acetonitrile, and an aliquot was separated by reversed-phase radio-HPLC [250 mm×4.6 mm column of Alltech (Deerfield, Ill.) Econosil C₁₈ (5 μm); flow rate of 1 mL/min; with radio-detection of the effluent (Flow-One-Beta Series A-1000, Radiomatic Corp., Meriden, Conn.)].

For product analyses using taxadiene, 5α-hydroxy taxadiene or 5α-acetoxy taxadiene as substrate, the following conditions were employed [solvent A: 97.99% H₂O with 2% CH₃CN and 0.01% H₃PO₄ (v/v); solvent B: 99.99% CH₃CN with 0.01% H₃PO₄ (v/v); gradient: 0-5 min at 100% (A), 5-15 min at 0-50% (B), 15-55 min at 50-100% (B), 55-65 min at 100% (B), 65-70 min at 0-100% (A), 70-75 min at 100% (A)].

For product analyses using 5α, 13α-dihydroxy taxadiene, 5α-acetoxy-10β-hydroxy taxadiene or 5α,9α,10β,13α-tetraacetoxy taxadiene (taxusin) as substrate, the following conditions were used [solvent A: 97.99% H₂O with 2% CH₃CN and 0.01% H₃PO₄ (v/v); solvent B: 99.99% CH₃CN with 0.01% H₃PO₄ (v/v); gradient: 0-5 min at 100% (A), 5-7 min at 100-92% (A), 7-40 min at 8-100% (B), 40-45 min at 100% (B), 45-50 min at 0-100% (A), 50-55 min at 100% (A)].

The HPLC eluant was collected in 1-min fractions, and the appropriate fractions containing the radiolabeled product were combined. The fractious were taken to dryness under N₂ and dissolved in a minimum volume of benzene for GC-MS analysis.

GC-MS analyses were performed on a Hewlett-Packard 6890 GC-MSD system using a ZB-5 capillary column [Phenomenex (Torrance, Calif.); 30-m length; 0.25-mm inner diameter; coated with a 0.25-μm film of phenyl (5%) polysiloxane]. Cool on-column injection was used, with He flow rate of 0.7 mL min and a temperature program from 40° C. to 320° C. at 20° C. min⁻¹. Spectra were recorded at 70 eV.

For preparative-scale conversions, to obtain sufficient product for NMR analysis, a substrate concentration of 250 μM was used, with incubations and product isolations performed as before. In this case, an additional normal-phase TLC purification step was employed [20 cm×20 cm×0.5 mm silica gel 60 F254 (Merck, Darmstadt, Germany); with toluene:acetone (3:1, v/v) as the developing solvent (product derived from 5α-acetoxy taxadiene), or toluene:hexane:acetone (3:7:10, v/v/v) as the developing solvent (product derived from 5α-acetoxy-10β-hydroxy taxadiene); detection by radio-monitoring]. The product (R_{f}˜0.5 in both cases) was eluted from the gel with CH₃CN and further purified by HPLC under the conditions described above. Scintillation counting and GC-MS analysis of the purified materials (>99%) confirmed the isolation of 200 μg of a putative 5α-acetoxy-dihydroxy taxadiene (obtained via the conversion of 5α-acetoxy-10β-hydroxy taxadiene) and of 300 μg of a putative 5α-acetoxy-hydroxy taxadiene (obtained via the conversion of 5α-acetoxy taxadiene).

D. Materials and Methods—NMR Spectroscopy.

All NMR spectra were recorded on a Varian Inova-500 NMR spectrometer operating at 18° C. using a very sensitive 5-mm pulsed-field-gradient ¹H indirect-detection probe. Samples were dissolved in C₆D₆ to a final concentration of about 200 μM. Homonuclear 2D-TOCSY was acquired using a z-filtered DIPSI mixing sequence, a 60-ms mixing time, 10-KHz spin-lock field, 16 repetitions, 256 (t₁)×2048 (t₂) complex points, and 7,000-Hz sweep in each dimension. Homonuclear 2D-ROESY was acquired using a z-filtered mixing sequence with a 350-ms mixing time, 4-KHz spin-lock field, 64 repetitions, 256 (t₁)×2048 (t₂) complex points, and 7,000-Hz sweep in each dimension. 2D-HSQC was acquired using 128 repetitions, 256 (t₁)×1024 (t₂) complex points, 7,000 Hz in F2 and 15,000 Hz in F1. The time between repetitions was 1.5 seconds for these experiments. Data were processed using Varian. VNMR software, version 6.1C. The final data size after forward linear-prediction in t₁ and zero-filling in both dimensions was 1024 (F1)×2048 (F2) complex points for all experiments.

E. Materials and Methods—Enzyme Characterization.

To monitor expression of the recombinant cytochrome P450 enzymes, immunoblot analysis was performed by taking advantage of the appended C-terminal His₆-tag. For this purpose, 50 μg of soluble or microsomal protein prepared by standard protocol was separated by SDS-PAGE (10% denaturing gel), transferred by wet transfer blotting to nitrocellulose, and immobilized by UV-crosslinking. A monoclonal penta-his-specific antibody (Qiagen, Valencia, Calif.) was employed as a primary antibody and alkaline phosphatase-conjugated AffiniPure goat anti-mouse IgG (Jackson ImmunoResearch, West Grove, Pa.), was used as a secondary antibody. The Qiagen protocols were used throughout with a his-size marker as a reference.

The pH optimum of this microsomal taxane hydroxylase was determined in 0.25 M Tris.HCl (pH 7-9) and phosphate (pH 5.5-8.5) buffers over intervals of 0.5 pH unit. Kₘ and Vᵣₑₗ values were determined under standard hydroxylase-assay conditions with substrate concentrations ranging from 2 to 100 μM for 5α-acetoxy-10β-hydroxy taxadiene and 5 to 200 μM for 5α-acetoxy taxadiene. The SPSS (Chicago, Ill.) Sigmaplot Enzyme Kinetics 1.10 software package was used with the Michaelis-Menten method, and the data reported are the means±standard errors of triplicate analyses.

F. Results and Discussion

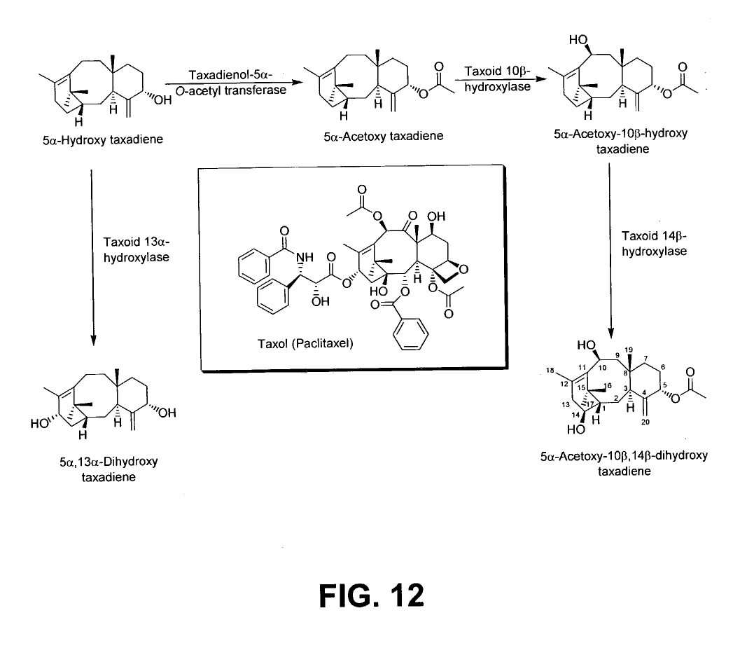

A central feature in the biosynthesis of Taxol and related advanced taxoids is the eight oxygenations of the taxane core (FIG. 12), all of which are considered to be mediated by cytochrome P450 monooxygenases. Following confirmation of expression of the target cytochrome P450 clones via immunoblot analysis of yeast microsomes isolated by standard protocol, test of function was conducted by in vivo feeding of the corresponding transformed yeast with [20-³H]5α-acetoxy-10β-hydroxy taxa-4(20),11(12)-diene or [20-³H]5α, 13α-dihydroxy taxa-4(20),11(12)-diene. Identical control feeding studies were carried out with yeast transformed with the vector harboring a β-galactosidase gene instead of the cytochrome P450 clone.

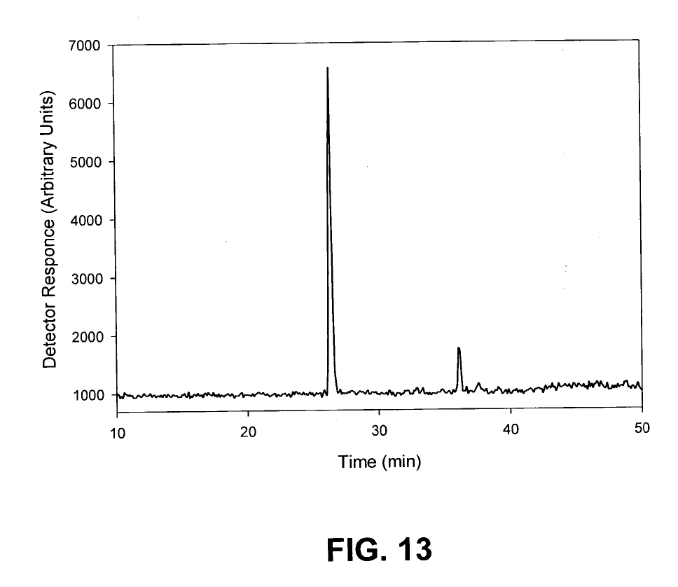

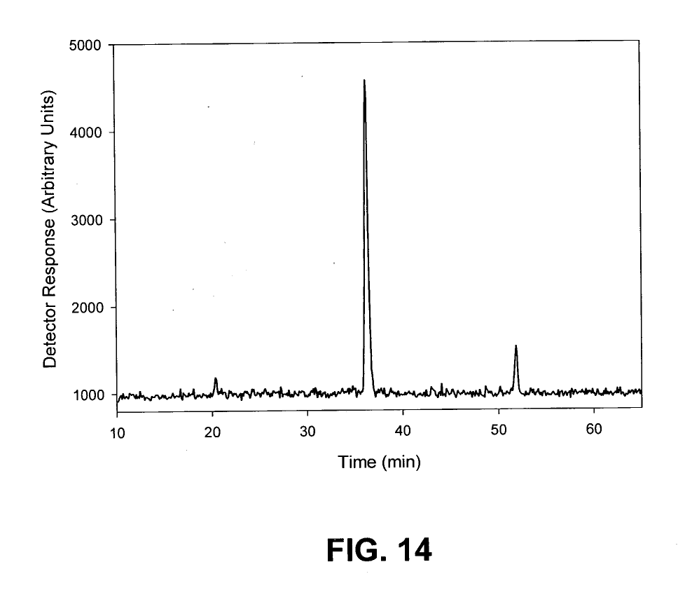

Only one clone (designated F72) of those tested appeared capable of converting 5α-acetoxy-10β-hydroxy taxadiene to a more polar labeled product, as demonstrated by radio-HPLC analysis of the extract of the reaction mixture (FIG. 13). Comparable feeding studies of the yeast cells that expressed clone F72 with radiolabeled taxa-4(5),11(12)-diene, 5α-hydroxy taxadiene, 5α,13α-dihydroxy taxadiene, and 5α,9α,10β,13α-tetracetoxy taxadiene (taxusin) showed no detectable turnover. However, 5α-acetoxy taxadiene was converted to a more polar product by these cells (FIG. 14) with slightly less efficiency than was 5α-acetoxy-10β-hydroxy taxadiene (cf FIG. 13). Control yeast cells that expressed β-galactosidase evidenced no detectable conversion of any of the taxoid substrates tested.

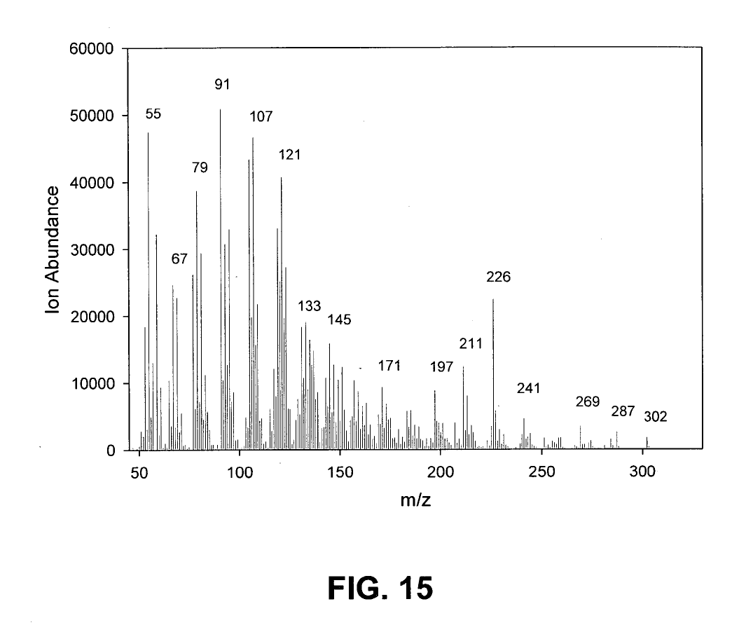

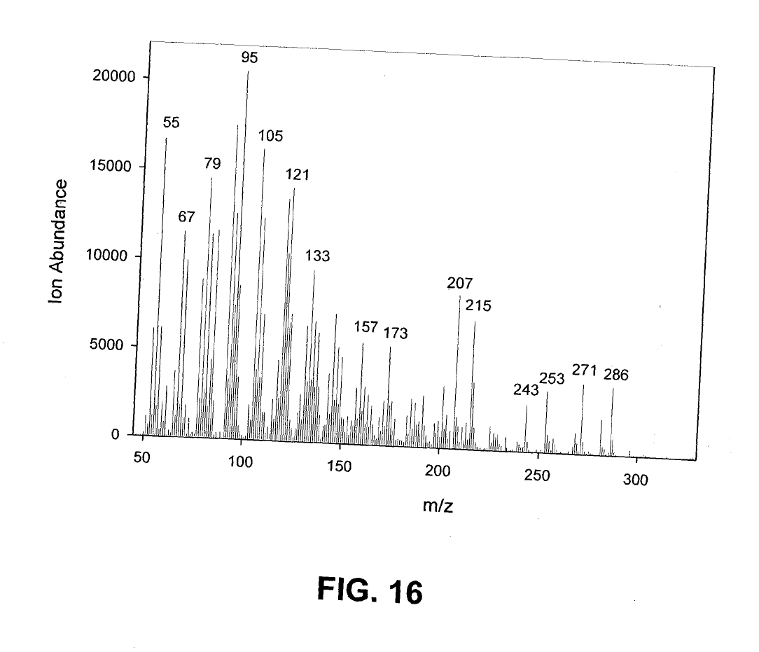

Preparative-scale conversion of 5α-acetoxy-10β-hydroxy taxadiene by the intact yeast harboring cytochrome P450 clone F72 allowed the isolation of ˜200 μg of the biosynthetic product by combination of TLC and HPLC (>99% pure as judged by GC-MS). The elution behavior of the product upon GC-MS analysis showed the polarity of a taxadien-triol monoacetate, and the spectrum revealed a high mass ion at m/z 302, corresponding to the loss of acetic acid from a parent ion of m/z 362 (i.e., P⁺−60) (FIG. 15). Additional diagnostic ions at m/z 287 (P⁺—CH₃COOH—CH₃) and m/z 269 (P⁺—CH₃COOH—CH₃—H₂O) were consistent with the putative identification of the biosynthetic product as an acetoxy-dihydroxy taxadiene.

The NMR analysis proceeded along the same lines as before for the structural definition of substituted taxadienes, with the challenge in this instance of determining the regiochemistry and stereochemistry of the newly appended hydroxyl function. Since only a limited amount of the biosynthetic product was available, and direct ¹³C measurements proved elusive, the analysis was restricted to ¹H-detected experiments that had greater intrinsic sensitivity. Despite this approach, it still was not possible to measure signals for the quaternary carbons.

Table 6 shows the complete ¹H NMR assignments along with their one-bond-correlated ¹³C assignments as measured indirectly by HSQC.

TABLE 6

| Position | Carbon δ, | α-Proton δ, | β-Proton δ, |
|---|---|---|---|
| number | ppm | ppm | ppm |
| 1 | 55.6 | — | 1.44 |
| 2 | 26.6 | 1.53 | 1.53 |
| 3 | 36.6 | 2.58 | — |
| 5 | 75.8 | — | 5.43 |
| 6 | 27.9 | 1.53 | 1.64 |
| 7 | 33.7 | 1.79 | 0.88 |
| 9 | 47.6 | 1.42 | 2.20 |
| 10 | 67.0 | 4.86 | — |
| 13 | 42.3 | 2.35 | 2.46 |
| 14 | 70.6 | 3.59 | — |
| 16 | 31.7 | 1.34 (exo) | — |
| 17 | 25.9 | 1.61 (endo) | — |
| 18 | 20.6 | 1.70 | — |
| 19 | 21.3 | — | 0.54 |
| 20 | 111.7 | 5.08 (exo) | — |
| | | 4.68 (endo) | — |
| acetate | 20.6 | 1.68 | — |

These assignments were consistent with structures of other known taxadienes bearing one or more oxygen functions. Thus, it was apparent that there were three oxy-methines (δ75.8, C5; δ5.43, H5), (δ67.0, C10; δ4.86, H10), and C14 (δ70.6, C14; δ3.59, H14). The 4(20) double-bond and exo-cyclic methylene at position 20 were intact (δ111.7, C20; δ5.08 and 4.68, H20) as expected for this taxadiene. Other characteristic signals were those observed for the H7 protons (δ1.79, H7_{α}; δ0.88, H7_{β}) and the H19 methyl (δ0.54). The H3 proton signal appears downfield (δ2.58) relative to the H1 signal that is buried in an envelope together with other signals (δ1.44). However, the C1 carbon signal stands out downfield (δ55.6) compared with that for C3 (δ36.6). The gem-dimethyls, H16 (δ1.34, exo) and H17 (δ1.61, endo), were also evident. The acetyl methyl and the C-19 methyl were nearly isochronous (Table 6). This NMR evidence indicates that a new acetoxy dihydroxy taxadiene had been isolated.

The homonuclear 2D-TOCSY supported the assignments made from heteronuclear HSQC data and allowed completion of additional regiochemical assignments. The H5 proton (δ5.43) was correlated strongly with H6 (δ1.64, δ1.54) and H7 (δ1.79, δ0.88) protons but had no appreciable coupling to either of the H20 signals (δ5.08, δ4.68) or with H3 (δ2.58), which was as expected in taxadiene derivatives. The H20 signals showed weak correlations to H3 (δ2.58) with extended correlations visible through H3 (δ2.58), H2 (δ1.53), and H1 (δ1.44). However, there was no correlation seen beyond H1 to other A-ring protons such as H14_{α}; which was not surprising since models of taxadiene indicated that these two protons were orthogonal. Correlations were observed between the H 14 signal and those for H 13 (δ2.46 and δ2.35) and for the C18 methyl (δ1.70). In contrast to the more extended spin correlations noted above, the H9 (δ2.20, δ1.42) and H10 (δ4.86) signals formed an isolated spin system. A correlation between the two gem-dimethyl signals (δ1.34, δ1.61) was observed, consistent with other taxadienes. The hydroxy protons were also identified in the TOCSY from correlations with their respective methines (C10-OH δ0.76 and C14-OH δ0.89).

A 2D-ROESY spectrum was used to confirm the regiochemical assignments already made and to assess the relative stereochemistry of the product. Although the 2D-TOCSY and 2D-HSQC suggested that a third heteroatom was introduced into the A-ring at C14, the regiochemistry and stereochemistry were confirmed only by using the ROESY data. Thus, the absence of a TOCSY correlation between H1 and the remainder of the A-ring protons was consistent with oxidation occurring at C14 from the β face, and was in contrast to the extended correlations between the A, B, and C-ring protons seen in several other taxadienes without the 14-β hydroxyl function. Models suggest that H-14_{α} and H1_{β} protons are nearly orthogonal, and so this will make the scalar coupling constant between them quite small. The ROESY spectrum supported this assignment. Correlations also were noted between the H14_{α} signal and the signals for H3, H13_{α}, and H2_{α}, as well as a weak correlation with H1_{β}, which all support the stereochemical assignment at C14.

Additional assignments were made by noting other prominent ROESY correlations. Several β-face protons, including H9_{β}, H2_{β}, H1 and the C19 methyl protons, interacted with one of the two gem-dimethyls, specifically C17 which must be assigned the endo position. The C17 methyl also correlates strongly with its twin in the C16exo position. The H13_{β} proton signal was the only other correlation noted with C16exo. The H10^{α} proton was exposed to more of the α-face of the taxadiene core and thus interacted with the C18 allylic methyl, H9_{α}, and also weakly with H7_{α}. The lack of any interaction between H10_{α} and either gem-dimethyl confirmed the presence of the expected C10_{β}-hydroxyl function.

ROESY also confirmed the relative stereochemistry of the acetate group at C5. Moderately strong correlations were observed between H5 and both H6 signals which were consistent with an equatorial orientation for H5. The ³J_{HH} coupling was quite small (<3 Hz) between H5 and all other scalar coupled partners, and was further evidence for the adopted equatorial orientation of H5. This still left open the question of whether the 5-OH was up (β) or down (α). Moderately strong n.O.e's were observed between H5 and H20exo, and between H5 and both H6 signals. No other significant n.O.e. correlations were observed between H5 and either face of the product. This led to the conclusion that H5 was on the β-face and in an equatorial configuration, and that the acetate group was on the α-face and in an axial configuration, as expected from the structure of the substrate. One other significant structural motif noted in taxadiene derivatives is the near occlusion of the H3 proton on the bottom (α-face) of the structure, due to the folding of the B-ring. This makes the H3 proton another useful probe for this face. Indeed, n.O.e. correlations were observed between H3, H⁷_{α}, H14_{α}, and the allylic methyl, H18.

By the combination of GC-MS and NMR analyses, the biosynthetic product was identified as 5α-acetoxy-10β, 14β-dihydroxy taxadiene, thereby confirming that the cytochrome P450 encoded by clone F72 was a taxoid 14β hydroxylase.

Preparative-scale conversion of 5α-acetoxy taxadiene by the intact yeast harboring cytochrome P450 clone F72 led to the isolation of ˜300 μg of a second biosynthetic product by combination of TLC and HPLC (>99% pure as judged by GC-MS). The elution behavior of this product upon GC-MS analysis suggested the polarity of a taxadien-diol monoacetate, and the spectrum revealed a high-mass ion at m/z 286, corresponding to the loss of acetic acid from a parent ion of m/z 346 (i.e., P⁺−60) (FIG. 5). Additional diagnostic ions at m/z 271 (P⁺—CH_{c}COOH—CH₃) and m/z 253 (P⁺—CH₃COOH—CH₃—H₂O) were consistent with the putative identification of the biosynthetic product as an acetoxy-hydroxy taxadiene.

The NMR analysis of this second product proceeded as before with comparable interpretation of the very similar spectra; the principal difference in this case relating to the characteristic resonances and correlations of the C10 α and β protons (2.7 and 2.1 ppm) found in other taxoids that are unsubstituted at this position. This second biosynthetic product, derived from 5α-acetoxy taxadiene, was identified as 5α-acetoxy-14β-hydroxy taxadiene, entirely consistent with the operation of a 14β-hydroxylase.

The pH optimum for the microsomal 14β-hydroxylase was determined to be near pH 7.5 (with a broad activity profile) in Tris-HCl buffer, similar to previously characterized cytochrome P450 taxoid hydroxylases. Kinetic evaluation of the taxane 14β-hydroxylase was carried out with both 5α-acetoxy taxadiene and 5α-acetoxy-10β-hydroxy taxadiene, and yielded a Kₘ value of 33±13 μM for the former and 55×14 μM for the latter; these values are similar to those for other enzymes of this family [13, 14, 21]. Vᵣₑₗ for the conversion of 5α-acetoxy taxadiene was only about 10% of that of 5α-acetoxy-10β-hydroxy taxadiene, indicating that, based on catalytic efficiency (Kₘ/Vᵣₑₗ), the latter was the preferred substrate by a factor of six.

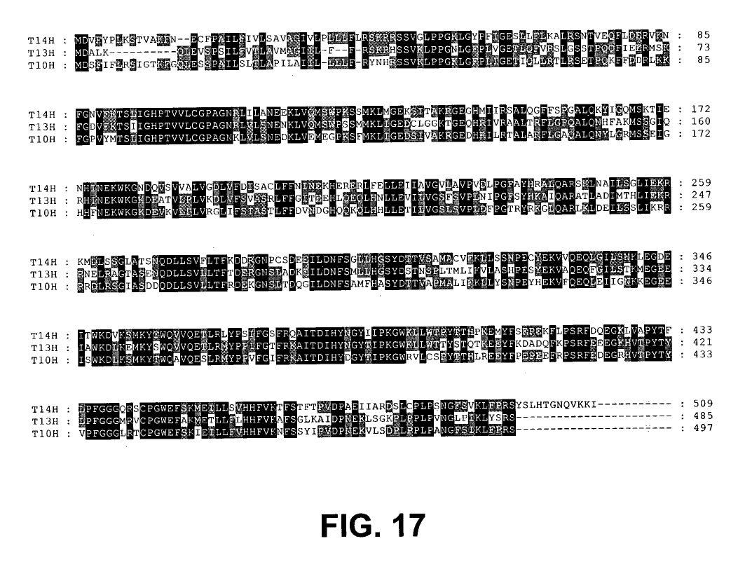

Clone F72 (GenBank Accession No. AY188177, to be released July, 2004; orf 1,530 bp SEQ ID NOS: 54 and 67) codes for a 509-residue protein, for which a molecular weight of 57,146 was calculated. This is in agreement with the size of the heterologously expressed enzyme observed by SDS-PAGE. Examination of the deduced amino acid sequence (FIG. 17) revealed several characteristics typical of cytochrome P450 enzymes, including the oxygen-binding domain, a highly conserved heme-binding motif with a PFG element (amino acids 436-438), and an absolutely conserved cysteine at position 444. In comparison to the sequences of most other cytochrome P450 enzymes, the N-terminus of the taxoid 140-hydroxylase contained a number of additional residues appended to the putative membrane anchor. Deduced-sequence comparisons of the taxoid 14β-hydroxylase with the previously identified taxoid 10β-hydroxyase and taxoid 13α-hydroxylase (FIG. 17) indicated an overall identity (in both cases) of 60%, and similarities in the 68-69% range. Interestingly, the 14β-hydroxylase, which fanctionalizes the taxane A-ring, displayed slightly more similarity to the 10β-hydroxylase (B-ring functionalization) than to the 13α-hydroxylase (A-ring functionalization), indicating that prediction of hydroxylation regiochemistry of these closely related cytochrome P450 clones based on homology is not possible.

Having illustrated and described the principles of the invention in multiple embodiments and examples, it should be apparent to those skilled in the art that the invention can be modified in arrangement and detail without departing from such principles. We claim all modifications coming within the spirit and scope of the following claims.

We claim:

1. A purified polypeptide, comprising a polypeptide having an amino acid sequence that is at least 75% identical to SEQ ID NO: 67, or a fragment thereof, wherein the polypeptide has oxygenase activity.

2. A purified polypeptide according to claim 1, wherein the polypeptide has an amino acid sequence that is at least 85% identical to SEQ ID NO: 67, or a fragment thereof.

3. A purified polypeptide according to claim 1, wherein the polypeptide has an amino acid sequence that is at least 95% identical to SEQ ID NO: 67, or a fragment thereof.

4. A purified polypeptide according to claim 1, wherein the polypeptide has an amino acid sequence that is at least 85% identical to SEQ ID NO: 67.

5. A purified polypeptide according to claim 1, wherein the polypeptide has an amino acid sequence that is at least 95% identical to SEQ ID NO: 67.

6. A purified polypeptide according to claim 1, wherein the polypeptide has an amino acid sequence comprising SEQ ID NO: 67, or a fragment thereof.

7. A purified polypeptide according to claim 6, wherein the polypeptide has an amino acid sequence comprising SEQ ID NO: 67.

8. A purified polypeptide according to claim 7, wherein the polypeptide has an amino acid sequence consisting essentially of SEQ ID NO: 67.

9. A specific binding agent that binds a polypeptide according to claim 1.

10. An isolated nucleic acid molecule encoding a polypeptide according to claim 1.

11. An isolated nucleic acid molecule according to claim 10, wherein the nucleic acid has a nucleotide sequence at least 75% identical to SEQ ID NO: 54, or a fragment thereof.

12. An isolated nucleic acid molecule according to claim 10, wherein the nucleic acid has a nucleotide sequence at least 85% identical to SEQ ID NO: 54, or a fragment thereof.

13. An isolated nucleic acid molecule according to claim 10, wherein the nucleic acid has a nucleotide sequence at least 95% identical to SEQ ID NO: 54, or a fragment thereof.

14. A recombinant nucleic acid molecule, comprising a promoter sequence operably linked to a nucleic acid molecule according to claim 10.

15. A cell transformed with a recombinant nucleic acid molecule according to claim 14.

16. A non-human transgenic organism comprising a recombinant nucleic acid molecule according to claim 14, wherein the transgenic organism is selected from the group consisting of plants, bacteria, insects, fungi, and mammals.

17. An isolated nucleic acid molecule that:
(a) hybridizes under low-stringency conditions with a nucleic acid probe, the probe comprising a nucleotide sequence according to SEQ ID 54, or a fragment thereof; and
(b) encodes a protein having oxygenase activity.

18. An oxygenase encoded by a nucleic acid molecule according to claim 17.

19. The oxygenase according to claim 18, wherein the oxygenase is a taxoid-14-beta hydroxylase.

20. A specific binding agent that binds to an oxygenase according to claim 18.

21. An isolated nucleic acid molecule that:
(a) has a nucleotide sequence at least 75% sequence identical to SEQ ID NO: 54; and
(b) encodes a polypeptide having oxygenase activity.

22. A method for isolating a nucleic acid sequence, comprising:
(a) hybridizing the nucleic acid sequence to at least 10 contiguous nucleotides of SEQ ID NO: 54; and
(b) identifying the nucleic acid sequence as one that encodes an oxygenase.

23. The method according to claim 22, wherein hybridizing the nucleic acid sequence is performed under low-stringency conditions.

24. A nucleic acid sequence isolated by the method of claim 22.

25. A purified oxygenase encoded by a nucleic acid sequence according to claim 24.

26. The purified oxygenase according to claim 25, wherein the oxygenase is a taxoid-14-beta-hydroxylase.

27. A specific binding agent that binds an oxygenase according to claim 25.

28. The method of claim 15, wherein the isolated nucleic acid sequence is isolated from the genus Taxus.

29. A purified protein having oxygenase activity, comprising a polypeptide having an amino acid that:
(a) comprises SEQ ID NO: 68;
(b) differs from the amino acid sequence specified in (a) by one or more conservative amino acid substitutions; or
(c) that is at least 70% identical to the sequence specified in either (a) or (b).

30. An isolated nucleic acid molecule encoding a protein according to claim 29.

31. A method for synthesizing a second intermediate in the Taxol® biosynthetic pathway, comprising:
(a) contacting a first intermediate with at least one oxygenase, wherein the oxygenase comprises an isolated polypeptide according to claim 1; and
(b) allowing the oxygenase to transfer at least one oxygen atom group to the first intermediate, wherein transfer of the at least one oxygen atom group yields the second intermediate in the Taxol® biosynthetic pathway.

32. The method according to claim 31, wherein the oxygenase is a taxoid-14-beta hydroxylase.

33. The method according to claim 31, wherein the oxygenase is produced by a transgenic oxygenase nucleic acid in a transgenic organism, and step (b) occurs in vivo.

34. A method for transferring an oxygen atom to a taxoid, comprising:
(a) contacting a taxoid with at least one oxygenase, wherein the oxygenase comprises an isolated polypeptide according to claim 1; and
(b) allowing the oxygenase to transfer an oxygen atom to the taxoid.

35. The method according to claim 34, wherein the oxygenase is produced by a transgenic oxygenase nucleic acid in a transgenic organism, and synthesis of the taxoid occurs in vivo.

36. The method of claim 30, wherein at least one paclitaxel molecule is produced.
